(19) United States
(12) Patent Application Publication
(10) Pub. No.: US 2008/0040826 A1
(43) Pub. Date: Feb. 14, 2008

(54) Stress Tolerant Transgenic Wheat Plant

(75) Inventors: Scott David McNeil, Kenmore (AU); Douglas Alan Chamberlain, Bibra Lake (AU); Robert Syndecombe Bower, St. Claire (NZ)

Correspondence Address: FOLEY AND LARDNER LLP;SUITE 500, 3000 K STREET NW, WASHINGTON, DC, 20007, US

(73) Assignee: Grain Biotech Australia Pty Ltd., South Perth (AU)

(21) Appl. No.: 11/659,228

(22) PCT Filed: Jul. 29, 2005
(86) PCT No.: PCT/AU05/01116

§ 371 Date: Aug. 9, 2007

(30) Foreign Application Priority Data
Aug. 3, 2004 (AU) 2004904326

(51) Int. Cl. A01H 5/00; A01H 1/00; C12N 15/82; C12N 15/87

(52) U.S. Cl. 800/289; 536/23.6; 800/278; 800/320.3

(57) ABSTRACT

The present invention relates to transgenic wheat plants. In particular, the present invention relates to a stress tolerant wheat plant, wherein said wheat plant has been transformed with a nucleic acid molecule, which codes for ornithine amino transferase (OAT).

This application is based on and claims the benefit of Australian provisional application 2004904326 filed 3 Aug. 2004.

### FIELD OF THE INVENTION

The present invention relates to transgenic wheat plants. In particular, the present invention relates to a transgenic wheat plant with increased tolerance to environmental stress, such as salt stress.

### BACKGROUND OF THE INVENTION

The threat of increased salinity to agriculture is increasing throughout the world at an alarming rate. With an increase in world population and a decrease in arable land it is essential to fully utilise plant biotechnology to increase crop production. In order to reduce the impact of salt stress on crop production there is a need for salt tolerant varieties of plants such as cereal plants.

There have been numerous attempts to breed plants with increased tolerance to salt stress. For example, conventional crossbreeding of wild species has produced new wheat varieties with some tolerance to salt stress. However, conventional crossbreeding is a slow process for generating new crop varieties, and limited germplasm resources for stress tolerance and incompatibility in crosses between distantly related plant species confer additional problems to conventional breeding techniques. Moreover, the salt tolerance of plants successfully produced by conventional crossbreeding is still relatively low with commercial varieties resistance to no more than 100 mM salt.

Salt stress is caused by a reduction in water potential in the cells of affected plants, and also by excess sodium ions, which impact on critical biochemical pathways in plant cells. Recently, research has turned to molecular approaches in order to develop crops with increased tolerance to salt stress. Accumulated experimental observations and theoretical considerations have suggested that a mechanism which may underlie the tolerance of plants to salt stress is the accumulation of compatible, low molecular weight osmolytes such as polyols/sugars, specific amino acids, and onium compounds.

Numerous studies have linked the accumulation of proline in plants to increased tolerance to salt, as well as increased tolerance to water deprivation, high and low temperature, toxicity of heavy metals, pathogen infections, anaerobiosis, nutrient deficiencies, atmospheric pollution and UV irradiation (see, for example, Stewart & Lee, 1974, Planta, 120: 279-289; Briens & Larher, 1982, Plant, Cell & Environ., 5: 287-292; Barnett & Naylor, 1966, Plant Physiol., 41: 1222-1230; Boggess et al., 1976, Aust. J. Plant Physiol., 3: 513-525; Jones et al., 1980, Aust. J. Plant Physiol., 7: 193-205; Katz & Tal, 1980, Z. Pflanzenphysiol. Bd., 98: 283-288; Treichel, 1986, Plant Physiol., 67: 173-181; Thomas et al., 1992, Plant Physiol. 98: 626-631). Studies reviewed by Hare and Cress (1997) have pointed out that during imposition of a stress on a plant, an increase in the proline level in the plant is linked to the amelioration of negative physiological effects. For example, analysis of experimental evidence collected from many studies suggests that proline accumulation may serve to protect plant cell membranes and polypeptides against the adverse effects of inorganic ions and temperature extremes.

The proline concentration in a plant cell can be increased by increasing the production of proline and/or by decreasing the degradation of proline. There are two pathways which produce proline in a plant cell. These are the glutamate pathway and the ornithine pathway. It has been suggested that proline is produced in young Arabidopsis thaliana plants from either glutamate or ornithine, while in mature plants, or when exposed to stress, the glutamate pathway usually dominates (see, for example, Roosens et al., 1998, Plant Physiol, 117: 263-271).

Several genes that encode enzymes involved in the biosynthesis of specific osmolytes, such as proline, have been introduced into the dicotyledonous plant tobacco. However, while regenerated tobacco plants did show partial tolerance to salt stress (see, for example, Tarczynski et al., 1993, Science, 259: 508-510; Kishor et al., 1995, Plant Physiol., 108:1387-1394; Lilius et al., 1966, Biotech., 14:177-180), these studies do not appear to have been continued.

More importantly, while some research with tobacco, a dicotyledonous plant, has been undertaken to date no genes involved in the biosynthesis of osmolytes like proline have been introduced into a wheat plant. Consequently, there is still a need for a commercial wheat plant that is capable of withstanding stress.

### SUMMARY OF THE INVENTION

Inventors have surprisingly found that the introduction into a wheat plant of a nucleic acid molecule encoding ornithine amino transferase (OAT) provides a transgenic wheat plant with an induced or increased tolerance to stress. Accordingly, in its most general form, the invention disclosed herein provides a stress tolerant wheat plant and a method for protecting said plants. The method utilises a nucleic acid molecule, which codes for ornithine amino transferase (OAT).

In a first aspect, the present invention provides a stress tolerant wheat plant, wherein said wheat plant has been transformed with a nucleic acid molecule, which codes for ornithine amino transferase (OAT).

In a second aspect, the present invention provides a method for protecting a wheat plant from stress, comprising the step of introducing a nucleic acid molecule into a wheat plant, which nucleic acid molecule codes for ornithine amino transferase (OAT).

In one embodiment the stress is selected from the group consisting of drought, salt, dehydration, heat, cold, freezing, water logging, wounding, mechanical stress, oxidative stress, ozone, high light, heavy metals, nutrient deprivation and toxic chemicals. More preferably, the stress is salt, frost or drought. Most preferably, the stress is the presence of more than 100 mM salt or temperature below 0° C.

The nucleic acid molecule may be cDNA, RNA, or a hybrid molecule thereof. Preferably the nucleic acid molecule is a cDNA molecule encoding ornithine amino transferase (OAT). Most preferably the cDNA molecule has a nucleotide sequence which is substantially that shown in FIG. 2 (SEQ ID NO:1) or biologically active fragment thereof.

The nucleic acid molecule may integrate into the host cell genome, or may exist as an extrachromosomal element.

The ornithine amino transferase (OAT) nucleic acid molecule may be isolated from any plant species. Preferably, the plant is Arabidopsis thaliana.

The wheat plant transformed by the ornithine amino transferase (OAT) nucleic acid molecule may be any variety of wheat plant. Preferably, the wheat plant is selected from the group consisting of Triticum aestivum, and Triticum durum.

In a third aspect, the present invention also provides a transgenic wheat plant, plant material, seeds or progeny thereof, comprising a nucleic acid molecule, which codes for ornithine amino transferase (OAT), wherein the expression of said nucleic acid molecule results in a transgenic plant, plant material, seeds or progeny thereof which is capable of growing in the presence of more than 100 mM salt.

In a fourth aspect, the present invention provides a nucleic acid construct comprising a promoter isolated from a plant and an ornithine amino transferase (OAT) gene as herein defined.

The promoter may be constitutive, ubiquitous, stress-inducible, tissue-specific or developmentally-controlled. Preferably the promoter is ubiquitin promoter.

In one embodiment, the construct is substantially the one shown in FIG. 1. However, it will be appreciated that modified and variant forms' of the constructs may be produced in vitro, by means of chemical or enzymatic treatment, or in vivo by means of recombinant DNA technology. Such constructs may differ from those disclosed, for example, by virtue of one or more nucleotide substitutions, deletions or insertions, but substantially retain a biological activity of the construct or nucleic acid molecule of this invention.

In another embodiment the transgenic wheat plant further comprises an endogenous proline degrading system which has been down regulated.

The transgenic plant may further comprise a polynucleotide, which encodes a selectable marker, and which is operably linked to the polynucleotide that encodes OAT, thereby facilitating selection of the transgenic wheat plant.

In another embodiment, the present invention provides food produced from a transgenic wheat plant of the invention.

In a fifth aspect, the present invention provides a method for producing a transgenic wheat plant with an induced or increased tolerance to salt, the method comprising the steps of:

a) transforming plant tissue or cell of a wheat plant with a nucleic acid molecule which codes for ornithine amino transferase (OAT);

b) regenerating the tissue or cell into a whole plant, and

c) expressing the OAT in the regenerated plant for a time and under conditions sufficient to induce or increase the tolerance of the plant to greater than 100 mM salt.

In another embodiment, the method further comprises the step of transforming the wheat plant with a polynucleotide encoding a selectable marker which is operably linked to the nucleic acid molecule which codes for ornithine amino transferase (OAT), thereby facilitating selection of the transgenic wheat plant.

In another embodiment, the method further comprises the step of down regulating the activity of an endogenous proline degrading system in the transgenic wheat plant.

### BRIEF DESCRIPTION OF THE FIGURES

FIG. 1 shows a nucleic acid construct comprising the ubiquitin promoter operably linked to OAT.

FIG. 2 shows the nucleotide sequence of OAT.

FIG. 3 shows the amino acid sequence of OAT.

FIG. 4 shows the high, medium and low salt tolerant levels for the line 2490.1. The transgenic line splits into 3 distinct groups.

FIG. 5 shows the impact on seed development for the transgenic plants (2490.1 and 2721.1) and their respective control varieties (Westonia and Carnamah) 13 days after a single frosting event.

### DEFINITIONS

The description that follows makes use of a number of terms used in recombinant DNA technology. Unless defined otherwise, all technical and scientific terms used herein have the meaning commonly understood by a person skilled in the art to which this invention belongs. The following references provide one of skill with a general definition of many of the terms used in this invention: Singleton, et al., Dictionary of Microbiology and Molecular Biology (2nd ed. 1994); The Cambridge Dictionary of Science and Technology (Walker ed., 1988); The Glossary of Genetics, 5^{th} Ed., Rieger, R., et al. (eds.), Springer Verlag (1991); and Hale & Marham, The Harper Collins Dictionary of Biology (1991). However, in order to provide a clear and consistent understanding of the specification and claims, including the scope given such terms, the following definitions are provided.

The term “cell” can refer to any cell from a plant, including but not limited to, somatic cells, gametes or embryos.

“Embryo” refers to a sporophytic plant before the start of germination. Embryos can be formed by fertilisation of gametes by sexual crossing or by selfing. A “sexual cross” is pollination of one plant by another. “Selfing” is the production of seed by self-pollination, i.e., pollen and ovule are from the same plant. The term “backcrossing” refers to crossing a F1 hybrid plant to one of its parents. Typically, backcrossing is used to transfer genes, which confer a simply inherited, highly heritable trait into an inbred line. The inbred line is termed the recurrent parent. The source of the desired trait is the donor parent. After the donor and the recurrent parents have been sexually crossed, F, hybrid plants which possess the desired trait of the donor parent are selected and repeatedly crossed (i.e., backcrossed) to the recurrent parent or inbred line.

Embryos can also be formed by “embryo somatogenesis” and “cloning.” Somatic embryogenesis is the direct or indirect production of embryos from either cells, tissues or organs of plants.

Indirect somatic embryogenesis is characterised by growth of a callus and the formation of embryos on the surface of the callus.

Direct somatic embryogenesis is the formation of an asexual embryo from a single cell or group of cells on an explant tissue without an intervening callus phase. Because abnormal plants tend to be derived from a callus, direct somatic embryogenesis is preferred.

The common term, “grain” is the endosperm present in the ovules of a plant.

The phrase “introducing a nucleic acid sequence” refers to introducing nucleic acid sequences by recombinant means, including but not limited to, Agrobacterium-mediated transformation, biolistic methods, electroporation, in planta techniques, and the like. The term “nucleic acids” is synonymous with DNA, RNA, and polynucleotides. A plant containing the introduced nucleic acid sequence is referred to here as an R, generation plant. R1 plants may also arise from cloning, sexual crossing or selfing of plants into which the nucleic acids have been introduced.

A “nucleic acid molecule” or “polynucleic acid molecule” refers herein to deoxyribonucleic acid and ribonucleic acid in all their forms, i.e., single and double-stranded DNA, cDNA, mRNA, and the like.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

A DNA sequence “corresponds” to an amino acid sequence if translation of the DNA sequence in accordance with the genetic code yields the amino acid sequence (i.e., the DNA sequence “encodes” the amino acid sequence).

One DNA sequence “corresponds” to another DNA sequence if the two sequences encode the same amino acid sequence.

Two DNA sequences are “substantially similar” when at least about 85%, preferably at least about 90%, and most preferably at least about 95%, of the nucleotides match over the defined length of the DNA sequences.

A “heterologous” region or domain of a DNA construct is an identifiable segment of DNA within a larger DNA molecule that is not found in association with the larger molecule in nature. Thus, when the heterologous region encodes a plant gene, the gene will usually be flanked by DNA that does not flank the plant genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide in vivo. A polyadenylation signal and transcription termination sequence will usually be located 3′ to the coding sequence.

Polynucleotide “homologs” refers to DNAs or RNAs and polymers thereof in either single- or double-stranded form containing known analogues of natural nucleotides, which have similar binding properties as the reference nucleic acid and are metabolised in a manner similar to naturally occurring nucleotides.

“Transgenic plants” are plants into which a nucleic acid has been introduced through recombinant techniques, eg., nucleic acid-containing vectors. A “vector” is a nucleic acid composition which can transduce, transform or infect a cell, thereby causing the cell to express vector-encoded nucleic acids and, optionally, proteins other than those native to the cell, or in a manner not native to the cell. A vector includes a nucleic acid (ordinarily RNA or DNA) to be expressed by the cell. A vector optionally includes materials to aid in achieving entry of the nucleic acid into the cell, such as a retroviral particle, liposome, protein coating or the like. Vectors contain nucleic acid sequences that allow their propagation and selection in bacteria or other non-plant organisms. For a description of vectors and molecular biology techniques, see Current Protocols in Molecular Biology, Ausubel, et al., (eds.), Current Protocols, a joint venture between Greene Publishing Associates, Inc. and John Wiley & Sons, Inc., (through and including the 1998 Supplement) (Ausubel).

“Plasmids” are one type of vector which comprises DNA that is capable of replicating within a plant cell, either extra-chromosomally or as part of the plant cell chromosome(s), and are designated by a lower case “p” preceded and/or followed by capital letters and/or numbers. The starting plasmids herein are commercially available, are publicly available on an unrestricted basis, or can be constructed from such available plasmids by methods disclosed herein and/or in accordance with published procedures. In certain instances, as will be apparent to the ordinarily skilled worker, other plasmids known in the art may be used interchangeably with plasmids described herein.

The phrase “expression cassette” refers to a nucleic acid sequence within a vector, which is to be transcribed, and a control sequence to direct the expression. The term “control sequences” refers to DNA sequences necessary for the expression of an operably linked nucleotide coding sequence in a particular host cell. The control sequences suitable for expression in prokaryotes, for example, include origins of replication, promoters, ribosome binding sites, and transcription termination sites. The control sequences that are suitable for expression in eukaryotes, for example, include origins of replication, promoters, ribosome-binding sites, polyadenylation signals, and enhancers. One of the most important control sequences is the promoter.

A “promoter” is an array of nucleic acid control sequences that direct transcription of a nucleic acid. As used herein, a promoter includes necessary nucleic acid sequences near the start site of transcription, such as, in the case of a polymerase II type promoter, a TATA element.

A promoter also optionally includes distal enhancer or repressor elements, which can be located as much as several thousand base pairs from the start site of transcription. The promoter can either be homologous, i.e., occurring naturally to direct the expression of the desired nucleic acid or heterologous, i.e., occurring naturally to direct the expression of a nucleic acid derived from a gene other than the desired nucleic acid. Fusion genes with heterologous promoter sequences are desirable, e.g., for regulating expression of encoded proteins. A “constitutive” promoter is a promoter that is active in a selected organism under most environmental and developmental conditions. An “inducible” promoter is a promoter that is under environmental or developmental regulation in a selected organism.

Examples include promoters from plant viruses such as the 35S promoter from cauliflower mosaic virus (CaMV), as described in Odell et al., (1985), Nature, 313:810-812, and promoters from genes such as rice actin (McElroy et al., (1990), Plant Cell, 163-171); ubiquitin (Christensen et al., (1992), Plant Mol. Biol. 12:619-632; and Christensen, et al., (1992), Plant Mol. Biol. 18:675-689); pEMU (Last et al., (1991), Theor. Appl. Genet. 81:581-588); MAS (Velten et al., (1984), EMBO J. 3:2723-2730); and maize H3 histone (Lepetit et al., (1992), Mol. Gen. Genet. 231:276-285; and Atanassvoa et al., (1992), Plant Journal 2(3):291-300).

Additional regulatory elements that may be connected to the OAT polynucleotides for expression in plant cells include terminators, polyadenylation sequences, and nucleic acid sequences encoding signal peptides that permit localisation within a plant cell or secretion of, the protein from the cell. Such regulatory elements and methods for adding or exchanging these elements with the regulatory elements of the replicase gene are known, and include, but are not limited to, 3′ termination and/or polyadenylation regions such as those of the Agrobacterium tumefaciens nopaline synthase (nos) gene (Bevan et al., (1983), Nucl. Acids Res. 12:369-385); the potato proteinase inhibitor II (PINII) gene (Keil, et al., (1986), Nucl. Acids Res. 14:5641-5650; and An et al., (1989), Plant Cell, 1:115-122); and the CaMV 19S gene (Mogen et al., (1990), Plant Cell, 2:1261-1272).

Plant signal sequences, including, but not limited to, signal-peptide encoding DNA/RNA sequences which target proteins to the extracellular matrix of the plant cell (Dratewka-Kos et al., (1989), J. Biol. Chem. 264:4896-4900), the Nicotiana plumbaginifolia extension gene (DeLoose et al., (1991), Gene, 99:95-100), signal peptides which target proteins to the vacuole like the sweet potato sporamin gene (Matsuka et al., (1991), PNAS, 88:834) and the barley lectin gene (Wilkins et al., (1990), Plant Cell, 2:301-313), signal peptides which cause proteins to be secreted such as that of PRIb (Lind et al., (1992), Plant Mol. Biol. 18:47-53), or the barley alpha amylase (BAA) (Rahmatullah et al. (1989), Plant Mol. Biol. 12:119 and hereby incorporated by reference).

For the purposes of the present invention, the promoter sequence is bounded at its 3′ terminus by the translation start codon of a coding sequence, and extends upstream to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase.

An “exogenous” element is one that is foreign to the host cell, or is homologous to the host cell but in a position within the host cell in which the element is ordinarily not found.

“Digestion” of DNA refers to the catalytic cleavage of DNA with an enzyme that acts only at certain locations in the DNA. Such enzymes are called restriction enzymes or restriction endonucleases, and the sites within DNA where such enzymes cleave are called restriction sites. If there are multiple restriction sites within the DNA, digestion will produce two or more linearised DNA fragments (restriction fragments). The various restriction enzymes used herein are commercially available, and their reaction conditions, cofactors, and other requirements as established by the enzyme manufacturers are used. Restriction enzymes are commonly designated by abbreviations composed of a capital letter followed by other letters representing the micro-organism from which each restriction enzyme originally was obtained and then a number designating the particular enzyme. In general, about 1 μg of DNA is digested with about 1-2 units of enzyme in about 20 μl of buffer solution. Appropriate buffers and substrate amounts for particular restriction enzymes are specified by the manufacturer, and/or are well known in the art.

“Recovery” or “isolation” of a given fragment of DNA from a restriction digest typically is accomplished by separating the digestion products, which are referred to as “restriction fragments,” on a polyacrylamide or agarose gel by electrophoresis, identifying the fragment of interest on the basis of its mobility relative to that of marker DNA fragments of known molecular weight, excising the portion of the gel that contains the desired fragment, and separating the DNA from the gel, for example by electroelution.

“Ligation” refers to the process of forming phosphodiester bonds between two double-stranded DNA fragments. Unless otherwise specified, ligation is accomplished using known buffers and conditions with 10 units of T4 DNA ligase per 0.5 μg of approximately equimolar amounts of the DNA fragments to be ligated.

“Oligonucleotides” are short-length, single- or double-stranded polydeoxynucleotides that are chemically synthesised by known methods (involving, for example, triester, phosphoramidite, or phosphonate chemistry), such as described by Engels et al., (1989), Agnew. Chem. Int. Ed. Engl. 28:716-734. They are then purified, for example, by polyacrylamide gel electrophoresis.

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis, using two oligonucleotide primers capable of hybridising preferentially to a template nucleic acid. Typically, the primers used in the PCR method will be complementary to nucleotide sequences within the template at both ends of or flanking the nucleotide sequence to be amplified, although primers complementary to the nucleotide sequence to be amplified also may be used. Wang et al., in PCR Protocols, pp. 70-75 (Academic Press, 1990); Ochman et al., in PCR Protocols, pp. 219-227; Triglia et al., (1988), Nucl. Acids Res. 16:8186.

“PCR cloning” refers to the use of the PCR method to amplify a specific desired nucleotide sequence that is present amongst the nucleic acids from a suitable cell or tissue source, including total genomic DNA and cDNA transcribed from total cellular RNA. Frohman et al., (1988), Proc. Nat. Acad. Sci. USA, 85:8998-9002; Saiki et al., (1988), Science, 239:487-492; Mullis et al., (1987), Meth. Enzymol. 155:335-350.

The phrase “operably encodes” refers to the functional linkage between a promoter and a second nucleic acid sequence, wherein the promoter sequence initiates transcription of RNA corresponding to the second sequence.

The term “progeny” refers to the descendants of a particular plant (self-cross) or pair of plants (crossed or backcrossed). The descendants can be of the F1, the Fez, or any subsequent generation.

Typically, the parents are the pollen donor and the ovule donor which are crossed to make the progeny plant of this invention.

Parents also refer to F1 parents of a hybrid plant of this invention (the F2 plants). Finally, parents refer to a recurrent parent, which is backcrossed to hybrid plants of this invention to produce another hybrid plant of this invention.

The phrase “producing a transgenic plant” refers to producing a plant of this invention. The plant is generated through recombinant techniques, i.e., cloning, somatic embryogenesis or any other technique used by those of skill to produce plants.

“Integration” of the DNA may be effected using non-homologous recombination following mass transfer of DNA into the cells using microinjection, biolistics, electroporation or lipofection. Alternative methods such as homologous recombination, and or restriction enzyme mediated integration (REMI) or transposons are also encompassed, and may be considered to be improved integration methods.

A “clone” is a population of cells derived from a single cell or common ancestor by mitosis.

“Nucleic acid sequence homologs” refers to deoxyribonucleotides or ribonucleotides and polymers thereof in either single- or double-stranded form containing known analogues of natural nucleotides, which have similar binding properties as the reference nucleic acid and are metabolised in a manner similar to naturally occurring nucleotides.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., (1991), Nucleic Acid Res. 19: 5081; Ohtsuka et al., (1985), J. Biol. Chem. 260: 2605-2608; and Rossolini et al., (1994), Mol. Cell. Probes 8: 91-98). The term “nucleic acid” is used interchangeably with gene, cDNA, and mRNA encoded by a gene.

The term “amino acid sequence homolog” refers to a protein with a similar amino acid sequence. One of skill will realise that the critical amino acid sequence is within a functional domain of a protein. Thus, it may be possible for a homologous protein to have less than 40% homology over the length of the amino acid sequence, but greater than 90% homology in one functional domain. In addition to naturally occurring amino acids, homologs also encompass proteins in which one or more amino acid residue is an artificial chemical analog of a corresponding naturally occurring amino acid, as well as to naturally occurring proteins.

Amino acids may be referred to herein by either their commonly known three letter symbols or by the one-letter symbols recommended by the IUPAC-IUB Biochemical Nomenclature Commission.

Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids that encode identical or essentially identical amino acid sequences, or where the nucleic acid does not encode an amino acid sequence, to essentially identical sequences.

Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognise that particular nucleic acids in a codon (except AUG, which is ordinarily the only codon for methionine) can be modified to yield a functionally identical molecule. Accordingly, each silent variation of a nucleic acid, which encodes a polypeptide, is implicit in each described sequence.

As to amino acid sequences, one of skill will recognise that individual substitutions, deletions or additions to a nucleic acid, peptide, polypeptide, or protein sequence that alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(See, e.g., Creighton, PROTEINS (1984)).

As used herein, the terms “transformation” and “transfection” refer to the process of introducing a desired nucleic acid, such a plasmid or an expression vector, into a plant's cells, either in culture or in the organs of a plant by a variety of techniques used by molecular biologists. Accordingly, a cell has been “transformed” by exogenous DNA when such exogenous DNA has been introduced inside the cell wall. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eukaryotic cells, a stably transformed cell is one in which the exogenous DNA is inherited by daughter cells through chromosome replication. This stability is demonstrated by the ability of the eukaryotic cell to establish cell lines or clones comprised of a population of daughter cells containing the exogenous DNA.

Numerous methods for introducing foreign genes into plants are known and can be used to insert a modified nucleic acid into a plant host, including biological and physical plant transformation protocols. See, for example, Miki et al., (1993), “Procedure for Introducing Foreign DNA into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, micro-organism-mediated gene transfer such as Agrobacterium (Horsch et al., (1985), Science, 227:1229-31), electroporation, micro-injection, and biolistic bombardment.

Expression cassettes and vectors and in vitro culture methods for plant cell or tissue transformation and regeneration of plants are known and available. See, for example, Gruber et al., (1993), “Vectors for Plant Transformation” In: Methods in Plant Molecular Biology and Biotechnology, Glick and Thompson, eds. CRC Press, Inc., Boca Raton, pages 89-119.

The most widely utilised method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectfully, carry genes responsible for genetic transformation of plants. See, for example, Kado (1991), Crit. Rev. Plant Sci. 10: 1. Descriptions of the Agrobacterium vector systems and methods for Agrobacterium-mediated gene transfer are provided in Gruber et al., supra; Miki et al., supra; and Moloney et al., (1989), Plant Cell Reports, 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into host organisms show fidelity in gene expression with respect to tissue/organ specificity of the original coding sequence. See, e.g., Benfey and Chua (1989), Science, 244: 174-181. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. application Ser. No. 913,914, filed Oct. 1, 1986, as referenced in U.S. Pat. No. 5,262,306, issued Nov. 16, 1993 to Robeson et al.; and Simpson et al. (1986), Plant Mol. Biol. 6: 403-415 (also referenced in the '306 patent); all incorporated by reference in their entirety.

Once constructed, these plasmids can be placed into A. rhizogenes or A. tumefaciens and these vectors used to transform cells of plant species, which are ordinarily susceptible to salinity. Several other transgenic plants are also contemplated by the present invention including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledons, some gymnosperms, and a few monocotyledons (e.g. certain members of the Liliales and Arales) are susceptible to infection with A. tumefaciens. A. rhizogenes also has a wide host range, embracing most dicots and some gymnosperms, which includes members of the Leguminosae, Compositae and Chenopodiaceae. Alternative techniques, which have proven to be effective in genetically transforming plants, include particle bombardment and electroporation. See e.g. Rhodes et al., (1988), Science, 240: 204-207; Shigekawa and Dower, (1988), Bio/Techniques, 6: 742-751; Sanford et al., (1987), Particulate Science & Technology, 5:27-37; and McCabe, (1988), Bio/Technology, 6:923-926.

Once transformed, these cells can be used to regenerate transgenic plants, capable of withstanding environmental stress. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors and cultured under conditions, which promote plant regeneration. Roots or shoots transformed by inoculation of plant tissue with A. rhizogenes or A. tumefaciens, containing the gene coding for the gene of interest, can be used as a source of plant tissue to regenerate transgenic plants, either via somatic embryogenesis or organogenesis. Examples of such methods for regenerating plant tissue are disclosed in Shahin, (1985), Theor. Appl. Genet. 69:235-240; U.S. Pat. No. 4,658,082; Simpson et al., (1986), Plant Mol. Biol., 6: 403-415; and U.S. patent application Ser. Nos. 913,913 and 913,914, both filed Oct. 1, 1986, as referenced in U.S. Pat. No. 5,262,306, issued Nov. 16, 1993 to Robeson, et al.; the entire disclosures therein incorporated herein by reference.

Despite the fact that the host range for Agrobacterium-mediated transformation is broad, some major cereal crop species and gymnosperms have generally been recalcitrant to this mode of gene transfer, even though some success has recently been achieved in rice (Hiei et al., (1994), The Plant Journal, 6:271-282). Several methods of plant transformation have been developed as an alternative to Agrobacterium-mediated transformation and these are collectively referred to as direct gene transfer techniques.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes. (Sanford et al., (1987), Part. Sci. Technol. 5:27; Sanford, 1988, Trends Biotech, 6:299; Sanford, (1990), Physiol. Plant 79:206; Klein et al., (1992), Biotechnology 10:268).

Another method for physical delivery of DNA to plants is sonication of target cells as described in Zang et al., (1991), Bio/Technology, 9:996. Alternatively, liposome or spheroplast fusions have been used to introduce expression vectors into plants. See, for example, Deshayes et al., (1985), EMBO J. 4:2731; and Christou et al., (1987), PNAS USA, 84:3962. Direct uptake of DNA into protoplasts using CaCl₂ precipitation, polyvinyl alcohol or poly-L-ornithine, have also been reported. See, for example, Hain et al., (1985), Mol. Gen. Genet. 199:161; and Draper et al., (1982), Plant Cell Physiol. 23:451.

Electroporation of protoplasts and whole cells and tissues has also been described. See, for example, Donn et al., (1990), In: Abstracts of the VII^{th} Int'l. Congress on Plant Cell and Tissue Culture IAPTC, A2-38, page 53; D'Halluin et al., (1992), Plant Cell 4:1495-1505; and Spencer et al., (1994), Plant Mol. Biol. 24:51-61.

Alternatively, the DNA constructs are combined with suitable T-DNA flanking regions and introduced into a conventional Agrobacterium tumefaciens host vector. The virulence function of the Agrobacterium tumefaciens host directs the insertion of the construct and adjacent marker into the plant cell DNA when the bacteria infect the cell.

Microinjection techniques are known in the art and well described in the scientific and patent literature. The introduction of DNA constructs using polyethylene glycol precipitation is described in Paszkowski et al., 1984, EMBO J. 3: 2717. Electroporation techniques are described in Fromm et al., 1985, Proc. Nat'l. Acad. Sci. USA, 82: 5824. Ballistic transformation techniques are described in Klein et al., 1987, Nature 327: 70-73.

Agrobacterium tumefaciens-mediated transformation techniques, including disarming and use of binary vectors, are also well described in the scientific literature. See, for example Horsch et al., 1984, Science, 233: 496-498, and Fraley et al., 1983, Proc. Nat'l. Acad. Sci. USA, 80: 4803.

One preferred method of transforming plants of the invention is microprojectile bombardment. In this method target tissues are treated with osmoticum. Then modified OAT gene DNA is precipitated, and coated on to tungsten or gold microparticles. The microparticles are then loaded into microprojectile or biolistic device and the treated cells are bombarded (Bower et al., 1996).

### DETAILED DESCRIPTION OF THE INVENTION

All publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention employs, unless otherwise indicated, conventional molecular biology, plant biology, and recombinant DNA techniques within the skill of the art. Such techniques are well known to the skilled worker, and are explained fully in the literature. See, e.g., Maniatis, Fritsch & Sambrook, “Molecular Cloning: A Laboratory Manual” (1982); “DNA Cloning: A Practical Approach,” Volumes I and II (D. N. Glover, Ed., 1985); “Oligonucleotide Synthesis” (M. J. Gait, Ed., 1984); “Nucleic Acid Hybridization” (B. D. Hames & S. J. Higgins, eds., 1985); “Transcription and Translation” (B. D. Hames & S. J. Higgins, eds., 1984); B. Perbal, “A Practical Guide to Molecular Cloning” (1984), and Sambrook, et al., “Molecular Cloning: a Laboratory Manual” 12^{th} edition (1989).

It is understood that the invention is not limited to the particular materials and methods described, as these may vary. It is also to be understood that the terminology used herein is for the purpose of describing particular embodiments only, and it is not intended to limit the scope of the present invention which will be limited only by the appended claims. It must be noted that as used herein and in the appended claims, the singular forms “a”, “an”, and “the” include plural reference unless the context clearly dictates otherwise. Thus, for example, a reference to “a polynucleotide” includes a plurality of such polynucleotides, and a reference to “an enhancer element” is a reference to one or more enhancer elements. Although any materials and methods similar or equivalent to those described herein can be used to practice or test the present invention, the preferred materials and methods are now described.

One of the broadest aspects of the present invention contemplates the use of a transgene engineered so as to produce a transgenic wheat plant, which expresses an ornithine amino transferase (OAT) gene. Ornithine amino transferase (OAT) is an enzyme of the ornithine pathway, which produces proline in a plant cell. OAT catalyses the transfer of an amino group from ornithine to alpha-ketoglutarate, yielding glutamic-5-semi-aldehyde and glutamic acid. Accordingly, as used herein, the term “transgenic plant” is intended to refer to a plant that has incorporated therein an OAT polynucleotide sequence, including but not limited to polynucleotides which are perhaps not normally present, DNA sequences not normally transcribed into RNA or translated into a protein (“expressed”).

The term “wheat plant” as used herein, includes, for example, any plant found in the wheat family selected from the group consisting of Triticum aestivum, Triticum durum, Triticum aestivum var. westonia, Triticum monococcum, Triticum aegilopoides, Triticum turgidum, Triticum polonicum, Triticum carthlicum, Triticum dicoccum, Triticum paleocolchicum, Triticum aestivum var. carnamah and Triticum turanicum.

The term “transgene” as used herein refers to any polynucleotide sequence, which codes for an OAT polypeptide, which is introduced into the genome of a wheat plant cell by experimental manipulations. The transgene may be an “endogenous DNA sequence,” or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally-occurring sequence. The term “heterologous DNA sequence” refers to a nucleotide sequence which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes an endogenous DNA sequence, which contains some modification. Generally, although not necessarily, heterologous DNA encodes RNA and proteins that are not normally produced by the cell into which it is expressed. Examples of heterologous DNA include mutated wild-type genes (i.e., wild-type genes that have been modified such that they are no longer wild-type genes), reporter genes, transcriptional and translational regulatory sequences, selectable marker proteins (e.g., proteins which confer drug resistance), etc.

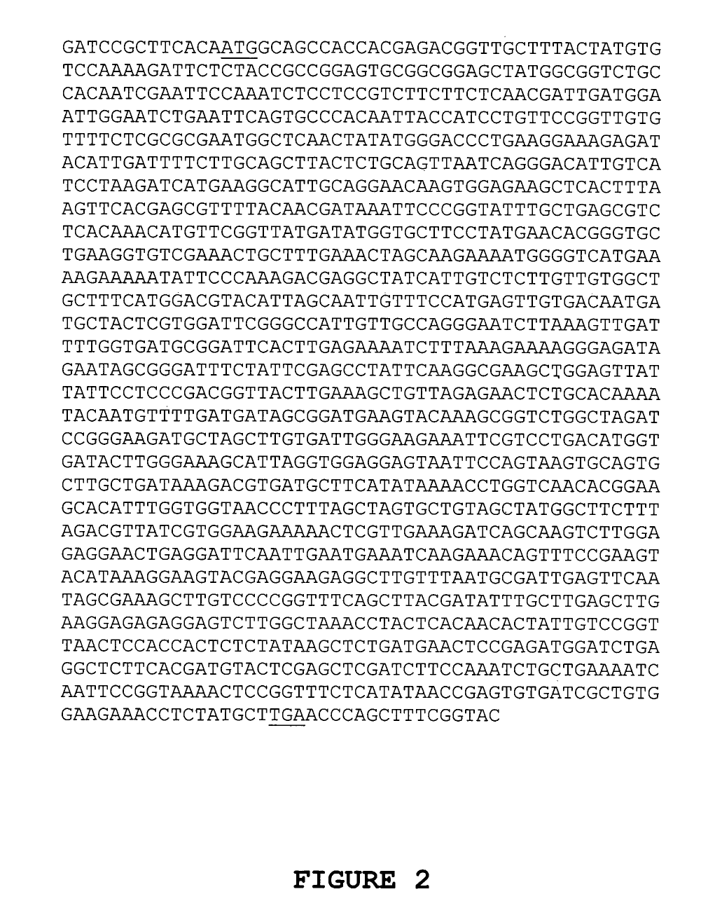

Thus, once an appropriate wheat host plant has been identified as discussed above, a transgene is constructed which comprises one or more OAT polynucleotides or functionally active fragments thereof. The term “functionally active,” when used in reference to the OAT polynucleotides of the present invention, refers to the paradigm in which an alteration to a nucleotide sequence does not necessarily affect the sequences ability to code for a polypeptide capable of performing substantially the same function as the unaltered “parent” polypeptide. For example, a nucleotide sequence may be truncated, elongated, or mutated in such a way that the polypeptide coded by the nucleotide sequence differs from the “parent” sequence, but still codes for a polypeptide that is capable of functioning in a substantially similar way to the “parent” molecule. Consequently, a functionally active derivative, analog, homolog or variant of the OAT polynucleotide of the present invention will have a nucleotide sequence which differs from the nucleotide sequence shown in FIG. 2 (SEQ ID NO:1), but the polypeptide coded for by the functionally active derivative, analog, homolog or variant is capable of displaying one or more known functional activities associated with the OAT polypeptides. Such modifications may be deliberate, as by site-directed mutagenesis, or may be spontaneous.

Synonyms of OAT (EC-Number 2.6.1.13) include L-ornithine: 2-oxo-acid aminotransferase, ornithine aminotransferase, ornithine-oxo-acid transaminase, aminotransferase ornithine-keto acid, L-ornithine 5-aminotransferase, L-ornithine aminotransferase, L-ornithine: alpha-ketoglutarate delta aminotransferase, ornithine 5-amino transferase, ornithine delta-transaminase, ornithine transaminase, ornithine-2-oxoacid aminotransferase, ornithine-alpha-ketoglutarate aminotransferase, ornithine-keto acid aminotransferase, ornithine-keto acid transaminase, ornithine ketoglutarate aminotransferase, ornithine-oxo acid aminotransferase, and ornithine: alpha-oxoglutarate transaminase.

It will be appreciated by those skilled in the art that a functionally active derivative, analog, homolog or variant of the OAT polynucleotide of the present invention can vary substantially outside regions of importance eg receptor binding sites; however, regions of high sequence conservation between OAT polynucleotides isolated from different virus species are likely to code for important regions such as receptor binding sites and the like. Accordingly, it is likely that mutations in these highly conserved regions will not generate functionally active derivatives, analogs, homologs or variants. For example, the conserved nucleotide sequences shown in Table 1 are likely to remain unchanged unless the changes are extremely conservative.

Sequences that are substantially similar can be identified in a Southern hybridisation experiment, for example under high, medium or low stringency conditions as defined for that particular system. Defining appropriate hybridisation conditions is within the skill of the art. See e.g., Sambrook et al., DNA Cloning, vols. I, II and III. Nucleic Acid Hybridization. However, ordinarily, “stringent conditions” for hybridisation or annealing of nucleic acid molecules are those that

(1) employ low ionic strength and high temperature for washing, for example, 0.015M NaCl/0.0015M sodium citrate/0.1% sodium dodecyl sulphate (SDS) at 50° C., or

(2) employ during hybridisation a denaturing agent such as formamide, for example, 50% (vol./vol.) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

An example of medium stringency conditions for hybridisation is the use of 50% formamide, 5×SSC (0.75M NaCl, 0.075M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulphate at 42° C., with washes at 42° C. in 0.2×SSC and 0.1% SDS.

By way of further example, and not intended as limiting, low stringency conditions include those described by Shilo and Weinberg in 1981 (Proc. Natl. Acad. Sci. USA, 78:6789-6792). When filters containing DNA are treated using these conditions they are usually pre-treated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridisations are carried out in the same solution with the following modifications: 0.02% PVP, 0.02% Ficoll, 0.2% BSA, 100 μg/ml salmon sperm DNA, 10% (wt./vol.) dextran sulphate, and 5-20×10⁶ cpm ³²P-labeled probe is used. Filters are incubated in hybridisation mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency, which may be used, are well known in the art (e.g., as employed for cross-species hybridisation).

The OAT polynucleotides, functionally active derivatives, analogs or variants of the invention can be produced by various methods known in the art. For example, cloned OAT polynucleotides can be modified by any of numerous strategies known in the art (See, for example, Maniatis, T., 1990, Molecular Cloning, A Laboratory Manual, 2^{nd} ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro.

Additionally, the OAT encoding polynucleotide sequences can be mutated in vitro or in vivo, to create or destroy functional regions or create variations in functional regions and/or form new restriction endonuclease sites or destroy pre-existing ones, to facilitate further in vitro modification. Any technique for mutagenesis known in the art can be used, including, but not limited to, chemical mutagenesis, in vitro site-directed mutagenesis (Hutchinson et al., 1978, J. Biol. Chem. 253:6551).

Alternatively, polynucleotide variants of the OAT polynucleotides may result from degenerate codon substitutions or complementary sequences. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19: 5081; Ohtsuka et al., 1985, J. Biol. Chem. 260: 2605-2608; and Rossolini et al., 1994, Mol. Cell. Probes, 8: 91-98). Alternatively, a variant may be a polynucleotide which is substantially similar to SEQ ID NO: 1 (FIG. 2), or in which one or more nucleotides have been added, deleted or substituted, at the 3′ and/or 5′ end(s) of the polynucleotide, or within the polynucleotide.

In one embodiment, the OAT polynucleotides are double-stranded DNA molecules having at least 85% nucleotide sequence identity with SEQ ID NO 1. Once the appropriate transgene has been identified and either isolated or constructed, it is incorporated into an expression vector by standard techniques. Accordingly, the present invention also contemplates an expression vector comprising the transgene of the present invention. Thus, in one embodiment an expression vector is constructed which comprises an isolated and purified DNA molecule comprising a promoter operably linked to the coding region for the OAT, which coding region is operatively linked to a transcription-terminating region, whereby the promoter drives the transcription of the coding region. The coding region may include a segment or sequence encoding the OAT gene. The DNA molecule comprising the expression vector may also contain a plant intron, and may also contain other plant elements such as sequences encoding untranslated sequences (UTL's) and sequences which act as enhancers of transcription or translation.

Preferred plant transformation vectors include, but are not limited to, those derived from a Ti plasmid of Agrobacterium tumefaciens, as well as those disclosed, e.g., by Herrera-Estrella (1983), Bevan (1983), Klee (1985) and Eur. Pat. Appl. No. EP 0120516 (each specifically incorporated herein by reference).

As the expression vectors of the present invention are preferably used to transform a wheat plant, a promoter is selected that has the ability to drive expression in that particular species of plant. Promoters that function in different plant species are also well known in the art. Promoters useful in expressing the polypeptide in plants are those which are inducible, viral, synthetic, or constitutive as described (Odell et al., 1985 supra), and/or temporally regulated, spatially regulated, and spatio-temporally regulated. Preferred promoters include the enhanced CaMV35S promoters, and the FMV35S promoter.

The expression of a gene which exists in double-stranded DNA form localised to the plant nuclear genome involves transcription of messenger RNA (mRNA) from the coding strand of the DNA by an RNA polymerase enzyme, and the subsequent processing of the mRNA primary transcript inside the nucleus. A region of DNA referred to as the “promoter” regulates transcription of DNA into mRNA. The DNA comprising the promoter is represented by a sequence of bases that signals RNA polymerase to associate with the DNA and to initiate the transcription of mRNA using one of the DNA strands as a template to make a corresponding strand of RNA. The particular promoter selected should be capable of causing sufficient transcription of the OAT coding sequence to result in the substantial protection from environmental stress in the resultant plant.

OAT polynucleotides of the present invention can be driven by a variety of promoters in plant tissues. Promoters can be near constitutive (i.e. they drive transcription of the transgene in all tissue), such as the CaMV35S promoter, the 1′- or 2′-promoter derived from T-DNA of Agrobacterium tumefaciens, or tissue-specific or developmentally specific. Enhanced or duplicate versions of the CaMV35S and FMV35S promoters are particularly useful in the practice of this invention (Kay et al., 1987; Rogers, U.S. Pat. No. 5,378,619).

Alternatively, the plant promoter may be under environmental control. Such promoters are referred to here as “inducible” promoters. Examples of environmental conditions that may effect transcription by inducible promoters include pathogen attack, anaerobic conditions, or the presence of light.

Preferably, a promoter that is capable of directing strong expression is used. Such promoters include, but are not limited to, the maize ubiquitin promoter described in Christensen and Quail (1996), the rice actin promoter as described in McElroy D, Blowers AD Jenes B and Wu R (1990), the commelina mosaic virus promoter as described in Medberry S L, Lockhart B E L and Olszewskine (1992).

Those skilled in the art will recognise that there are a number of promoters, which are active in plant cells, and have been described in the literature. Such promoters may be obtained from plants or plant viruses and include, but are not limited to, the nopaline synthase (NOS) and octopine synthase (OCS) promoters (which are carried on tumour-inducing plasmids of A. tumefaciens), the cauliflower mosaic virus (CaMV) 19S and 35S promoters, the light-inducible promoter from the small subunit of ribulose 1,5-bisphosphate carboxylase (ssRUBISCO, a very abundant plant polypeptide), the rice Act1 promoter and the Figwort Mosaic Virus (FMV) 35S promoter. All of these promoters have been used to create various types of DNA constructs which have been expressed in plants (see e.g., McElroy et al., 1990, U.S. Pat. No. 5,463,175).

In addition, it may also be preferred to bring about expression of the OAT polynucleotide by using plant-integrating vectors containing a tissue-specific promoter.

Specific target tissues may include the leaf, stem, root, tuber, seed, fruit, etc., and the promoter chosen should have the desired tissue and developmental specificity. Therefore, promoter function should be optimised by selecting a promoter with the desired tissue expression capabilities and approximate promoter strength, and selecting a transformant, which produces the desired level of stress resistance in the target tissues. This selection approach from the pool of transformants is routinely employed in expression of heterologous structural genes in plants since there is variation between transformants containing the same heterologous gene due to the site of gene insertion within the plant genome (commonly referred to as “position effect”). In addition to promoters which are known to cause transcription (constitutive or tissue-specific) of DNA in plant cells, other promoters may be identified for use in the current invention by screening a plant cDNA library for genes which are selectively or preferably expressed in the target tissues, then determining the promoter regions.

Other exemplary tissue-specific promoters are corn sucrose synthetase 1 (Yang et al., 1990), corn alcohol dehydrogenase 1 (Vogel et al., 1989), corn light harvesting complex (Simpson, 1986), corn heat shock protein (Odell et al., 1985 supra), pea small subunit RuBP carboxylase (Poulsen et al., 1986; Cushmore et al., 1983), Ti plasmid mannopine synthase (McBride and Summerfelt, 1989), Ti plasmid nopaline synthase (Langridge et al., 1989), petunia chalcone isomerase (Van Tunen et al., 1988), bean glycine rich protein 1 (Keller et al., 1989), CaMV 35S transcript (Odell et al., 1985 supra) and Potato patatin (Wenzler et al., 1989) promoters. Preferred promoters are the cauliflower mosaic virus (CaMV 35S) promoter and the S-E9 small subunit RuBP carboxylase promoter.

The promoters used in the DNA constructs of the present invention may be modified, if desired, to affect their control characteristics. For example, the CaMV35S promoter may be ligated to the portion of the ssRUBISCO gene that represses the expression of ssRUBISCO in the absence of light, to create a promoter which is active in leaves but not in roots. For purposes of this description, the phrase “CaMV35S” promoter thus includes variations of CaMV35S promoter, e.g., promoters derived by means of ligation with operator regions, random or controlled mutagenesis, etc. Furthermore, the promoters may be altered to contain multiple “enhancer sequences” to assist in elevating gene expression. Kay et al. (1987) has reported examples of such enhancer sequences.

A transgenic plant of the present invention produced from a plant cell transformed with a tissue specific promoter can be crossed with a second transgenic plant developed from a plant cell transformed with a different tissue specific promoter to produce a hybrid transgenic plant that shows the effects of transformation in more than one specific tissue.

The RNA produced by a DNA construct of the present invention may also contain a 5′ non-translated leader sequence (5′UTL). This sequence can be derived from the promoter selected to express the gene, and can be specifically modified so as to increase translation of the mRNA. The 5′ non-translated regions can also be obtained from viral RNAs, from suitable eukaryotic genes, or from a synthetic gene sequence. The present invention is not limited to constructs wherein the non-translated region is derived from the 5′ non-translated sequence that accompanies the promoter sequence. One plant gene leader sequence for use in the present invention is the petunia heat shock protein 70 (hsp70) leader (Winter et al., 1988).

5′ UTL's are capable of regulating gene expression when localised to the DNA sequence between the transcription initiation site and the start of the coding sequence. Compilations of leader sequences have been made to predict optimum or sub-optimum sequences and generate “consensus” and preferred leader sequences (Joshi, 1987). Preferred leader sequences are contemplated to include those which comprise sequences predicted to direct optimum expression of the linked structural gene, i.e. to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants, and in maize in particular, will be most preferred. One particularly useful leader may be the petunia HSP70 leader.

For optimised expression an intron may also be included in the DNA expression construct. Such an intron is typically placed near the 5′ end of the mRNA in untranslated sequence. This intron could be obtained from, but not limited to, a set of introns consisting of the maize heat shock protein (HSP) 70 intron (U.S. Pat. No. 5,424,412; 1995), the rice Act1 intron (McElroy et al., 1990), the Adh intron 1 (Callis et al., 1987), or the sucrose synthase intron (Vasil et al., 1989).

The 3′ non-translated region of the genes of the present invention which are localised to the plant nuclear genome also contain a polyadenylation signal which functions in plants to cause the addition of adenylate nucleotides to the 3′ end of the mRNA. RNA polymerase transcribes a nuclear genome coding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Examples of preferred 3′ regions are (1) the 3′ transcribed, non-translated regions containing the polyadenylation signal of Agrobacterium tumour-inducing (Ti) plasmid genes, such as the nopaline synthase (NOS) gene and (2) the 3′ ends of plant genes such as the pea ribulose-1,5-bisphosphate carboxylase small subunit gene, designated herein as E9. (Fischhoff et al., 1987). Constructs will typically include the OAT polynucleotides along with a 3′ end DNA sequence that acts as a signal to terminate transcription and, in constructs intended for nuclear genome expression, allow for the polyadenylation of the resultant mRNA. The most preferred 3′ elements are contemplated to be those from the nopaline synthase gene of A. tumefaciens (nos 3′ end) (Bevan et al., 1983), the terminator for the T7 transcript from the octopine synthase gene of A. tumefaciens, and the 3′ end of the protease inhibitor I or II genes from potato or tomato. Regulatory elements such as TMV OMEGA element (Gallie et al., 1989), may further be included where desired.

Transcription enhancers or duplications of enhancers could be used to increase expression. These enhancers often are found 5′ to the start of transcription in a promoter that functions in eukaryotic cells, but can often be inserted in the forward or reverse orientation 5′ or 3′ to the coding sequence. Examples of enhancers include elements from the CaMV 35S promoter, octopine synthase genes (Ellis et al., 1987), the rice actin gene, and promoter from non-plant eukaryotes (e.g., yeast; Ma et al., 1988).

In certain embodiments of the invention, the use of internal ribosome binding sites (IRES) elements are used to create multigene, or polycistronic, messages. IRES elements are able to bypass the ribosome-scanning model of 5′ methylated Cap dependent translation and begin translation at internal sites (Pelletier and Sonenberg, 1985). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together; each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

The choice of which expression vector and ultimately to which promoter the OAT polynucleotide is operatively linked depends directly on the host cell to be transformed. These are well known limitations inherent in the art of constructing recombinant DNA molecules. However, a vector useful in practising the present invention is capable of directing the expression of the OAT coding region to which it is operatively linked.

The vector comprising the OAT sequence will also typically comprise a marker gene, which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon, or phosphinothricin (the active ingredient in bialaphos and Basta).

Typical vectors useful for expression of genes in higher plants are well known in the art and include vectors derived from the tumour-inducing (Ti) plasmid of A. tumefaciens described (Rogers et al., 1987). However, several other plant integrating vector systems are known to function in plants including pCaMVCN transfer control vector described (Fromm et al., 1985). pCaMVCN (available from Pharmacia, Piscataway, N.J.) includes the CaMV35S promoter.

In one embodiment, the vector used to express the OAT polynucleotide includes a selection marker that is effective in a plant cell. In another embodiment, the genes coding for the OAT polynucleotide and/or selection marker are on two or more separate vectors. Selection markers can be drug resistance selection markers or metabolic selection markers. One preferred drug resistance marker is the gene whose expression results in kanamycin resistance; i.e. the chimeric gene containing the nopaline synthase promoter, Tn5 neomycin phosphotransferase II (nptII) and nopaline synthase 3′ non-translated region described (Rogers et al., 1988).

Means for preparing expression vectors are well known in the art. Expression (transformation) vectors used to transform plants and methods of making those vectors are described in U.S. Pat. Nos. 4,971,908, 4,940,835, 4,769,061 and 4,757,011 (each of which is specifically incorporated herein by reference). Those vectors can be modified to include a coding sequence in accordance with the present invention.

A variety of methods have been developed to operatively link DNA to vectors via complementary cohesive termini or blunt ends. For instance, complementary homopolymer tracts can be added to the DNA segment to be inserted and to the vector DNA. The vector and DNA segment are then joined by hydrogen bonding between the complementary homopolymeric tails to form recombinant DNA molecules.

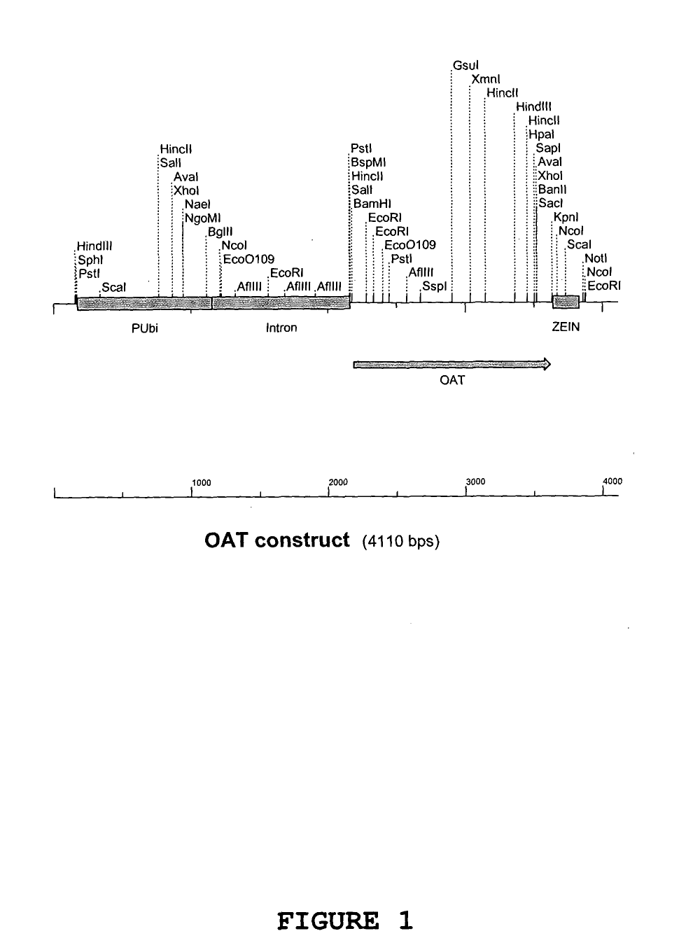

In one embodiment double-stranded DNA coding for the OAT shown in FIG. 2 (SEQ ID No. 1), is ligated to the ubiquitin promoter and the zein terminator to form an expression vector termed “pGBA2”, which is shown in FIG. 1.

A wheat plant transformed with an expression vector of the present invention is also contemplated. A transgenic plant derived from such a transformed or transgenic cell is also contemplated. Those skilled in the art will recognise that a chimeric plant gene containing a structural coding sequence of the present invention can be inserted into the genome of a plant by methods well known in the art. Such methods for DNA transformation of plant cells include Agrobacterium-mediated plant transformation, the use of liposomes, transformation using viruses or pollen, electroporation, protoplast transformation, gene transfer into pollen, injection into reproductive organs, injection into immature embryos and particle bombardment. Each of these methods has distinct advantages and disadvantages. Thus, one particular method of introducing genes into a particular plant strain may not necessarily be the most effective for another plant strain, but it is well known which methods are useful for a particular plant strain.

There are many methods for introducing transforming DNA segments into cells, but not all are suitable for delivering DNA to plant cells. Suitable methods are believed to include virtually any method by which DNA can be introduced into a cell, such as infection by A. tumefaciens and related Agrobacterium strains, direct delivery of DNA such as, for example, by PEG-mediated transformation of protoplasts (Omirulleh et al., 1993), by desiccation/inhibition-mediated DNA uptake, by electroporation, by agitation with silicon carbide fibres, by acceleration of DNA coated particles, etc. In certain embodiments, acceleration methods are preferred and include, for example, microprojectile bombardment and the like.

Technology for introduction of DNA into cells is well-known to those of skill in the art. Four general methods for delivering a gene into cells have been described: (1) chemical methods (Graham and van der Eb, 1973); (2) physical methods such as microinjection (Capecchi, 1980), electroporation (Wong and Neumann, 1982; Fromm et al., 1985) and the gene gun (Johnston and Tang, 1994; Fynan et al., 1993); (3) viral vectors (Clapp, 1993; Lu et al., 1993; Eglitis and Anderson, 1988a; 1988b); and (4) receptor-mediated mechanisms (Curiel et al., 1991; 1992; Wagner et al., 1992).

The application of brief, high-voltage electric pulses to a variety of animal and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

The introduction of DNA by means of electroporation is well known to those of skill in the art. To effect transformation by electroporation, one may employ either friable tissues such as a suspension culture of cells, or embryogenic callus, or alternatively, one may transform immature embryos or other organised tissues directly. One would partially degrade the cell walls of the chosen cells by exposing them to pectin-degrading enzymes (pectolyases) or mechanically wounding in a controlled manner, rendering the cells more susceptible to transformation. Such cells would then be recipient to DNA transfer by electroporation, which may be carried out at this stage, and transformed cells then identified by a suitable selection or screening protocol dependent on the nature of the newly incorporated DNA.

A further advantageous method for delivering transforming DNA segments to plant cells is microprojectile bombardment. In this method, particles may be coated with nucleic acids and delivered into cells by a propelling force. Exemplary particles include those comprised of tungsten, gold, platinum, and the like. Using these particles, DNA is carried through the cell wall and into the cytoplasm on the surface of small metal particles as described (Klein et al., 1987; Klein et al., 1988; Kawata et al., 1988). The metal particles penetrate through several layers of cells and thus allow the transformation of cells within tissue explants.

An advantage of microprojectile bombardment, in addition to it being an effective means of reproducibly stably transforming plant cells, is that neither the isolation of protoplasts (Cristou et al., 1988) nor the susceptibility to Agrobacterium infection is required. An illustrative embodiment of a method for delivering DNA into plant cells by acceleration is a Biolistics Particle Delivery System, which can be used to propel particles coated with DNA or cells through a screen, such as a stainless steel or Nytex screen, onto a filter surface covered with the plant cultured cells in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing damage inflicted on the recipient cells by projectiles that are too large.

For the bombardment, cells in suspension are preferably concentrated on filters or solid culture medium. Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus which express the exogenous gene product 48 hours post-bombardment often range from 1 to 10 and average 1 to 3.

In bombardment transformation, one may optimise the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearised DNA or intact supercoiled plasmids. It is believed that pre-bombardment manipulations are especially important for successful transformation of immature plant embryos.

Accordingly, it is contemplated that one may desire to adjust several of the bombardment parameters in small-scale studies to fully optimise the conditions. One may particularly wish to adjust physical parameters such as gap distance, flight distance, tissue distance, and helium pressure. One may also minimise the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

The methods of particle-mediated transformation are well known to those of skill in the art. U.S. Pat. No. 5,015,580 (specifically incorporated herein by reference) describes the transformation of soybeans using such a technique.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described (Fraley et al., 1985; Rogers et al., 1987). The genetic engineering of cotton plants using Agrobacterium-mediated transfer is described in U.S. Pat. No. 5,004,863 (specifically incorporated herein by reference); like transformation of lettuce plants is described in U.S. Pat. No. 5,349,124 (specifically incorporated herein by reference); and the Agrobacterium-mediated transformation of soybean is described in U.S. Pat. No. 5,416,011 (specifically incorporated herein by reference). Further, the integration of the Ti-DNA is a relatively precise process resulting in few rearrangements. The region of DNA to be transferred is defined by the border sequences, and intervening DNA is usually inserted into the plant genome as described (Spielmann et al., 1988; Jorgensen et al., 1987).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate construction of vectors capable of expressing various polypeptide-coding genes. The vectors described (Rogers et al., 1987), have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

Agrobacterium-mediated transformation of leaf disks and other tissues such as cotyledons and hypocotyls appear to be limited to plants that Agrobacterium naturally infects. Agrobacterium-mediated transformation is most efficient in dicotyledonous plants. Few monocots appear to be natural hosts for Agrobacterium, although transgenic plants have been produced in asparagus using Agrobacterium vectors as described (Bytebier et al., 1987). Other monocots recently have also been transformed with Agrobacterium. Included in this group are corn (Ishida et al. 1996) and rice (Cheng et al. 1998).

A transgenic plant formed using Agrobacterium transformation methods typically contains a single gene on one chromosome. Such transgenic plants can be referred to as being heterozygous for the added gene. However, inasmuch as use of the word “heterozygous” usually implies the presence of a complementary gene at the same locus of the second chromosome of a pair of chromosomes, and there is no such gene in a plant containing one added gene as here, it is believed that a more accurate name for such a plant is an independent segregant, because the added, exogenous gene segregates independently during mitosis and meiosis.

An independent segregant may be preferred when the plant is commercialised as a hybrid, such as corn. In this case, an independent segregant containing the gene is crossed with another plant, to form a hybrid plant that is heterozygous for the gene of interest.

An alternate preference is for a transgenic plant that is homozygous for the added OAT polynucleotide; i.e. a transgenic plant that contains two added genes, one gene at the same locus on each chromosome of a chromosome pair.

A homozygous transgenic plant can be obtained by sexually mating (selfing) an independent segregant transgenic plant that contains a single added gene, germinating some of the seed produced and analysing the resulting plants produced for gene of interest activity and Mendelian inheritance indicating homozygosity relative to a control (native, non-transgenic) or an independent segregant transgenic plant.

Two different transgenic plants can be mated to produce offspring that contain two independently segregating added, exogenous genes. Selfing of appropriate progeny can produce plants that are homozygous for both added, exogenous genes that encode a polypeptide of interest. Back-crossing to a parental plant and out-crossing with a non-transgenic plant are also contemplated.

Transformation of plant protoplasts can be achieved using methods based on calcium phosphate precipitation, polyethylene glycol treatment, electroporation, and combinations of these treatments (see e.g., Potrykus et al., 1985; Lorz et al., 1985; Fromm et al., 1985; Uchimiya et al., 1986; Callis et al., 1987; Marcotte et al., 1988).

Application of these systems to different plant germplasm depends upon the ability to regenerate that particular plant variety from protoplasts. Illustrative methods for the regeneration of cereals from protoplasts are described (see, e.g., Fujimura et al., 1985; Toriyama et al., 1987; Yamada et al., 1986; Abdullah et al., 1986).

To transform plant germplasm that cannot be successfully regenerated from protoplasts, other ways to introduce DNA into intact cells or tissues can be utilised. For example, regeneration of cereals from immature embryos or explants can be effected as described (Vasil, 1988).

DNA can also be introduced into plants by direct DNA transfer into pollen as described (Zhou et al., 1983; Hess, 1987). Expression of polypeptide coding genes can be obtained by injection of the DNA into reproductive organs of a plant as described (De La Pena et al., 1987). DNA can also be injected directly into the cells of immature embryos and introduced into cells by rehydration of desiccated embryos as described (Neuhaus et al., 1987; Benbrook et al., 1986).

After effecting delivery of exogenous OAT polynucleotides to recipient wheat cells, the next step to obtain the transgenic wheat plants of the present invention generally concerns identifying the transformed cells for further culturing and plant regeneration. As mentioned herein, in order to improve the ability to identify transformants, one may desire to employ a selectable or screenable marker gene as, or in addition to, the OAT polynucleotide. In this case, one would then generally assay the potentially transformed cell population by exposing the cells to a selective agent or agents, or one would screen the cells for the desired marker gene trait.

An exemplary embodiment of methods for identifying transformed cells involves exposing the transformed cultures to a selective agent, such as a metabolic inhibitor, an antibiotic, herbicide or the like. Cells that have been transformed and have stably integrated a marker gene conferring resistance to the selective agent used will grow and divide in culture. Sensitive cells will not be amenable to further culturing. One example of a preferred marker gene confers resistance to glyphosate. When this gene is used as a selectable marker, the putatively transformed cell culture is treated with glyphosate. Upon treatment, transgenic cells will be available for further culturing while sensitive, or non-transformed cells, will not. This method is described in detail in U.S. Pat. No. 5,569,834, which is specifically incorporated herein by reference. Another example of a preferred selectable marker system is the neomycin phosphotransferase (nptII) resistance system by which resistance to the antibiotic kanamycin is conferred, as described in U.S. Pat. No. 5,569,834 (specifically incorporated herein by reference). Again, after transformation with this system, transformed cells will be available for further culturing upon treatment with kanamycin, while non-transformed cells will not. Yet another preferred selectable marker system involves the use of a gene construct conferring resistance to paromomycin. Use of this type of a selectable marker system is described in U.S. Pat. No. 5,424,412 (specifically incorporated herein by reference).

Another preferred selectable marker system involves the use of the genes contemplated by this invention. In particular, cells transformed with the OAT polynucleotide or functional equivalents will develop salt tolerance. Plant cells which have had a recombinant DNA molecule introduced into their genome can thus be selected from a population of cells which failed to incorporate a recombinant molecule by growing the cells and isolating cells which are resistant to high levels of salt.

It is further contemplated that combinations of screenable and selectable markers will be useful for identification of transformed cells. In some cell or tissue types a selection agent, such as glyphosate or kanamycin, may either not provide enough killing activity to clearly recognise transformed cells or may cause substantial non-selective inhibition of transformants and non-transformants alike, thus causing the selection technique to not be effective. It is proposed that selection with a growth inhibiting compound, such as glyphosate at concentrations below those that cause 100% inhibition followed by screening of growing tissue for expression of a screenable marker gene such as a gene that codes for kanamycin resistance would allow one to recover transformants from cell or tissue types that are not amenable to selection alone. It is proposed that combinations of selection and screening may enable one to identify transformants in a wider variety of cell and tissue types.

The development or regeneration of plants from either single plant protoplasts or various explants is well known in the art (Weissbach and Weissbach, 1988). This regeneration and growth process typically includes the steps of selection of transformed cells, culturing those individualised cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a polypeptide of interest introduced by Agrobacterium from leaf explants can be achieved by methods well known in the art such as described (Horsch et al., 1985). In this procedure, transformants are cultured in the presence of a selection agent and in a medium that induces the regeneration of shoots in the plant strain being transformed as described (Fraley et al., 1983). In particular, U.S. Pat. No. 5,349,124 (specification incorporated herein by reference) details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art.

Preferably, the regenerated plants are self-pollinated to provide homozygous transgenic plants, or pollen obtained from the regenerated plants is crossed to seed-grown plants of agronomically important lines. These lines can be either inbred or out bred lines. Conversely, pollen from plants of those important lines is used to pollinate regenerated plants. A transgenic plant of the present invention containing a desired polypeptide is cultivated using methods well known to one skilled in the art.

In one embodiment, a transgenic plant of this invention thus has an increased amount of genes for OAT mRNA. A preferred transgenic plant is an independent segregant and can transmit these genes and their activities to its progeny. A more preferred transgenic plant is homozygous for the OAT polynucleotide, and transmits these to its entire offspring on sexual mating. Seed from a transgenic plant may be grown in the field or greenhouse, and resulting sexually mature transgenic plants are self-pollinated to generate true breeding plants. The progeny from these plants become true breeding lines that are evaluated for expression of the OAT transgene.

It is contemplated that in some instances the genome of a transgenic plant will have been augmented through the stable introduction of one or more OAT transgenes, either native, synthetically modified, or mutated. In some instances, more than one transgene will be incorporated into the genome of the transformed host plant cell. Such is the case when more than one OAT-encoding DNA segments are incorporated into the genome of such a plant. In certain situations, it may be desirable to have one, two, three, four, or even more OAT genes (either native or recombinantly engineered) incorporated and stably expressed in the transformed transgenic plant.

In one embodiment of the present invention the transgenic wheat plants having increased OAT polypeptide will have induce or increased tolerance to stress. The term “tolerance” covers the range of protection from a delay to complete inhibition of alteration in cellular metabolism, reduced cell growth and/or cell death caused by the stress condition. Preferably, the transgenic wheat plants of the present invention are tolerant to stress conditions in the sense that the wheat plants are capable of growing substantially normally under conditions where the corresponding wild-type wheat plant shows reduced growth, metabolism, viability, productivity and/or male or female sterility.

As used herein, “stress tolerance” refers to the capacity to grow and produce biomass during stress, the capacity to reinitiate growth and biomass production after stress, and the capacity to survive stress. The term “stress tolerance” also covers the capacity of the plant to undergo its developmental program during stress in a similar manner to a plant under non-stressed conditions, for example, to switch from dormancy to germination and from vegetative to reproductive phase under stressed conditions in a similar manner to a plant under non-stressed conditions. Methodologies to determine plant growth or response to stress include, but are not limited to height measurements, leaf area, plant water relations, ability to flower, and ability to generate progeny and yield or any other methodology known to the person skilled in the art.

The term “stress” includes biotic stress, such as stress caused by a pathogen (including viruses, bacteria, fungi, insects and nematodes) and combinations of these, as well as environmental stress, particularly since adverse environmental conditions may allow a pathogen to overcome the natural tolerance of a plant to stress.

The term “pathogen” means those organisms that have a negative effect on the physiological state of the plant. Examples of pathogens include viruses, bacteria, fungi, parasites and pests such as nematodes and insects, which are able to exert a negative effect on the physiological state of the plant.

The term “environmental stress” has been defined in different ways in the prior art and largely overlaps with the term “osmotic stress”. Holmberg & Bulow, 1998, (Trends Plant Sci. 3, 61-66) for instance define different environmental stress factors which result in abiotic stress. Salt, drought, heat, chilling and freezing are all described as examples of conditions which induce osmotic stress. The term “environmental stress” as used in the present specification refers to any adverse effect on metabolism, growth or viability of the cell, tissue, seed, organ or whole plant which is produced by a non-living or non-biological environmental stress. More particularly, it also encompasses environmental factors such as salt stress, water stress (flooding, water logging, drought, dehydration), anaerobic (low level of oxygen, CO₂ etc.), aerobic stress, osmotic stress, temperature stress (hot/heat, cold, freezing, frost) or nutrient deprivation, pollutant stress (heavy metals, toxic chemicals), ozone, high light, and combinations of these.

In one preferred embodiment the transgenic wheat plants of the present invention have increased capacity to withstand salt stress. The terms “salt-stress”, “salinity-induced stress”, or similar terms refer to any stress which is associated with or induced by elevated concentrations of salt and which result in a perturbation in the osmotic potential of the intracellular or extracellular environment of a cell. In particular, the salt tolerant wheat plants of the present invention are capable of exhibiting at least 350% increase in seed yield relative to untransformed “wild-type” wheat plants in the presence of at least 150 mM NaCl.

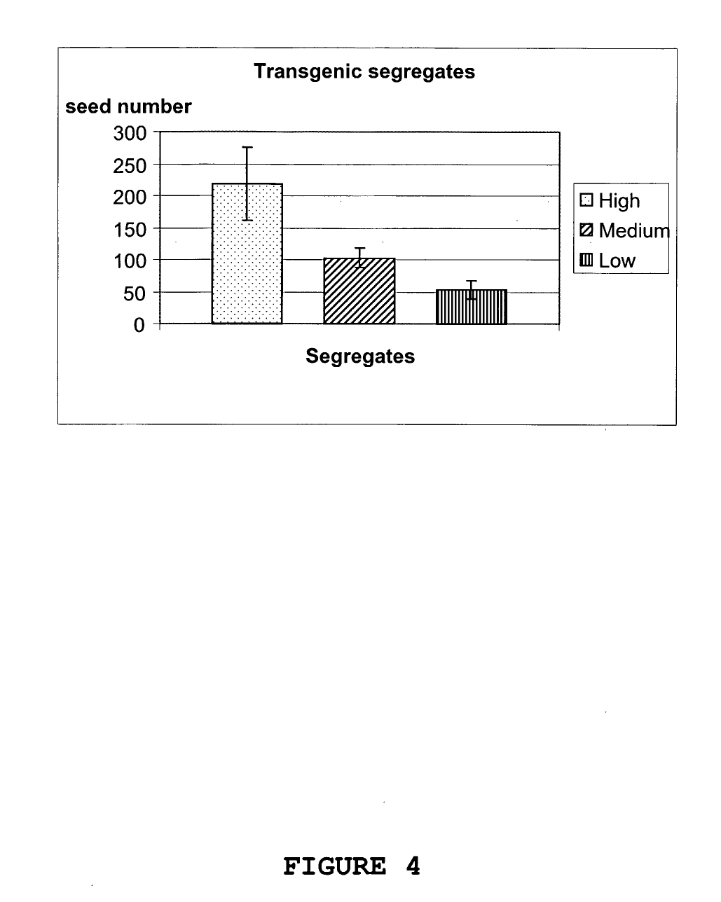

Using for example the procedure outlined in Example 6 infra transgenic plants can be tested in a hydroponics system at 150 mM salt against a commercial wheat cultivar such as Westonia. For example, 10 transgenic plants of the salt tolerant line 2490.1 segregated into three levels of salt tolerance (see FIG. 4). The highest group of 2490.1 had greater than 3.5 fold higher seed number, seed weight, tiller number and head number per plant than Westonia and the low salt tolerance transgenic group.

In one embodiment, the present invention provides food produced from a plant of the invention. As used herein, “food” means any substance that can be metabolised by an organism to give energy and build tissue, and includes both liquid and solid food.

Throughout the specification, the word “comprise” and variations of the word, such as “comprising” and “comprises”, means “including but not limited to” and is not intended to exclude other additives, components, integers or steps.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the use of a specific OAT gene in two wheat varieties, it will be clearly understood that the findings herein are not limited to this source of OAT genes or these wheat varieties.

### EXAMPLE 1

### Ornithine Aminotransferase (OAT) Gene

The OAT gene was isolated from Arabidopsis thaliana into a binary vector. For transformation into wheat the gene was amplified by PCR amplification and transferred into vector pGBA2 using BamH1 and Kpn1 restriction sites.

The primers for amplification of the OAT gene with restrictions sites BamH1 (5′ end) and Kpn1 (3′ end) where as follows:

Forward primer -
5′ GCATGGATCCGCTTCACAATGGCAGCAGCCACCACG

The BamH1 site is underlined and the start codon is bolded.

Reverse primer -
5′ GCATGGTACCGAAAGCTGGGTTCAAGCATAGAGG

The Kpn1 site is underlined and the stop codon is bolded.

The primers used to identify the Arabidopsis OAT gene in transformed wheat were as follows.

| | |
|---|---|
| Forward primer - | 5′ GAGTTGTGACAATGATGCTACTCGTGG |
| Reverse primer - | 5′ CGAGTACATCGTGAAGAGCCTCAGATCC |

The length of the predicted PCR product was 770 bp fragment.

Amplification of the OAT gene with BamH1 and Kpn1 restriction sites was with Pfu DNA polymerase (Promega). The reagents and procedure used were as outlined in the instruction sheet accompanying the Pfu. The PCR product was run on a 1% agarose gel and compared to a 1 kb plus DNA molecular ladder. Amplified product was purified from agarose gel using Ultraclean™ DNA Purification Kit (Geneworks) in accordance with the manufacturer's instructions. Briefly, the band of interest was excised from the gel, 3 gel volumes of Ultra-salt was added and the mixture incubated at 65° C. for 5 min to dissolve the agarose. 5-7 μL Ultra-Bind silica matrix was added, the solution mixed thoroughly and the tube incubated at room temperature for 5 min to bind DNA. The Bresa-Bind/DNA matrix was pelleted by centrifugation at 20,800 g for 5 sec, the supernatant discarded and the pellet resuspended in 1 ml of Ultra-Wash solution by vortexing for 10 sec. The tube was then centrifuged for 5 sec and all traces of supernatant removed by aspiration. The DNA was eluted from the Ultra-Bind by resuspending the pellet in 2 volumes of distilled water, incubating at room temperature for 5 min, centrifuged for 1 min at 20,800 g and removing the supernatant containing the DNA to a new tube.

Amplification of a fragment of the OAT gene to screen for wheat plants containing the gene was carried out as follows: The ingredients of the PCR were 4 mM MgCl₂, 10 mM Tris-HCL, 50 mM KCl, 0.75 mM dNTP and 0.2 pmol of each primer. The reaction conditions were 94° C. for 3 min then 35 cycles of 94 oC for 30 sec, 60° C. for 30 sec and 72° C. for 2 min. The reaction was the held at 72° C. for 7 min before a holding temperature of 15° C. The PCR reaction was then run on a 1% agarose gel and compared with a 1 kb plus DNA molecular ladder.

### EXAMPLE 2

### Cloning of the Oat Gene into pGBA2 Vector

The OAT gene product with BamH1 and Kpn1 restrictions sites from Example 1 was cut with BamH1 and Kpn1 restriction enzymes and ligated into the pGBA2 vector also cut with BamH1 and Kpn1 between the Ubiquitin promoter and zein terminator. The Ubiquitin promoter, OAT gene and Zein terminator make up the OAT construct (FIG. 1). E. coli strain DH10b competent cells were transformed with the ligation mixture and plated onto LB agar containing ampicillin to select for E. coli cells transformed with pGBA2. Transformed E. coli colonies were then tested for the presence of the OAT gene within the pGBA2 plasmid by PCR amplification of a 770 bp fragment of the OAT gene. The presence of the OAT gene was confirmed by restriction digest and match the pattern with the predicted pattern of fragment sizes.

### EXAMPLE 3

### Sequencing the OAT Gene

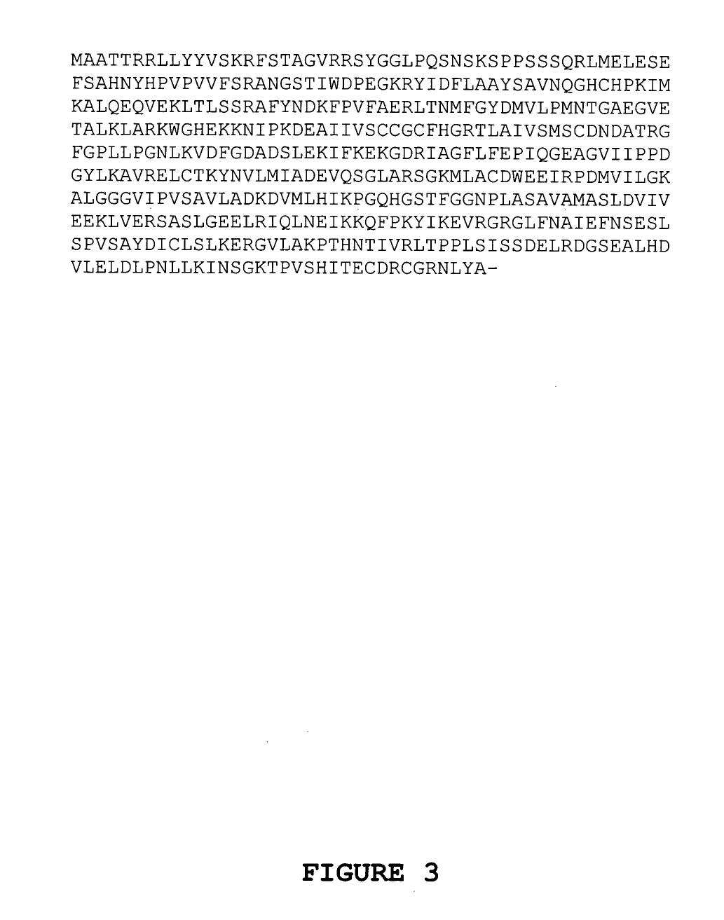

Sequencing was carried out on one clone identified in Example 2 using an automated sequencer ABI377 (Applied Biosystems Industries) in accordance with the manufacturer's instructions. The sequencing was performed with the forward and reverse primers from Example 1 that generate the 770 bp fragment plus another internal primer (5′ ACAATTGCTAATGTACGTCC). The SeqEd™ version 1.0.3 software (Applied Biosystems Industries) was used to analyse the raw sequence data and the MultAlin (Corpet, 1988) web based program for sequence alignment with the putative Arabidopsis OAT gene sequence from Genbank (NM 123987). FIG. 2 shows the nucleotide sequence obtained, while FIG. 3 shows the amino acid sequence of OAT.

### EXAMPLE 4

### Production of Transgenic Wheat Plants with OAT Construct

A population of transgenic wheat plants, containing OAT construct as described in Example 2, was generated using the following procedures.

Preparation of OAT Construct

The pGBA2, containing the OAT construct was digested with HaeII to produce a fragment containing the OAT construct and 177 bp of pGBA2 at the 5′ end of the construct and 283 bp of pGBA2 at the 3′ end of the construct (FIG. 1). The OAT construct was run on a 1% agarose gel and the DNA extracted from the gel as in Example 1. The DNA was resuspended in water to a concentration of 500 ng/μL.

Target Tissue

Wheat plants (cultivars: Westonia and Carnamah) were grown at 22-24° C. in a glasshouse. Seeds containing immature embryos were harvested at 11-15 days post-anthesis and surface sterilised. Immature embryos were excised and placed on MS (Murashige and Skoog, 1962) medium containing 2.5 mg/l 2,4 dichlorophenoxyacetic acid (2,4-D) prior to bombardment.

Microprojectile Bombardment

Osmoticum treatment of target tissues, DNA precipitation and microprojectile bombardment were performed as described for sugarcane (Bower et al., 1996) with the exception of the use of tungsten particles. Wheat tissues were bombarded with 50 μg of gold particles per bombardment. The linear fragments used for bombardment were CAH linear construct (Weeks et al., 2000), which encoded the cyanamide hydratase gene to degrade the cyanamide, in equal molecular concentrations with the linear OAT construct.

Selection

Following bombardment the embryos were placed on MS medium containing 2.5 mg/l 2,4-D for two weeks at 24° C. in the dark, transferred to the same medium plus 40 mg/l cyanamide (Sigma) for a further six weeks under the same culture conditions with fresh medium every two weeks. The tissue was then transferred to MS medium containing 0.1 mg/L 2,4-D and 40 mg/l cyanamide and transferred into the light for regeneration. After two weeks the tissue was transferred onto MS medium minus asparagine and glutamine and with 0.1 mg/L 2,4-D and 55 mg/l cyanamide (C2 medium). After two weeks the tissue was transferred to C2 medium without 2,4-D and with 65 mg/l of cyanamide (C3 medium). The healthy plantlets developed from the tissues are and then transferred into ½ MS media plus 65 mg/l cyanamide. Once they develop roots they are transferred into potting mix in the glasshouse.

### EXAMPLE 5

### Transgenic Analysis Based on PCR

In order to prove whether or not the plants generated in Example 4, did contain the transgene for the Arabidopsis OAT gene a fragment of the gene was amplified by PCR.

The method of DNA extraction from wheat leaf was as detailed in the instructions for the Nucleon PhytoPure™ (Amersham Biosciences) for extracting DNA from plants.

The PCR primers and conditions for the amplification of a 770 bp fragment are described in Examples 1 and 2.

Approximately 50% of the transgenics that came out of the selection system contained the OAT gene.

### EXAMPLE 6

### Salt Tolerance Screening Using a Saline Hydroponics System

Having identified the wheat plants with the OAT gene the transgenic TO plants were allowed to set seed and the seed then collected for the saline hydroponics screening.

From previous experiments 150 mM salt was found to significantly halt plant growth and cause early seed production.

The seeds were planted into rockwool cubes (one seed per cube) and allowed to germinate and grow in water until approximately 10 cm high. The plants were then transferred to perforated 41 pots containing basalt rock (2 cm blue metal). The pots were situated in a hydroponics continuous flow system with ½ Hoaglands solution filled to a height 2 cm below the top of the basalt rock. There were 5 plants per pot and 2 pots per transgenic line. The plants were allowed to adapt to the hydroponics system for 3 days before the solution was adjusted to 50 mM salt using NaCl:CaCl₂ at a 9:1 ratio respectively. The solution was adjusted up by 50 mM every 3 days until 150 mM was reached. The solution was then maintained at 150 mM salt. The reservoir tank was regularly adjusted with ½ Hoaglands solution to account for evaporation and nutrient uptake by the plants.

Once the plants had completed their life cycle they were harvested for phenotypic measurements. These measurements included tiller number per plant, head number per plant, seed weight per plant and seed number per plant.

The transgenic line 2490.1 showed salt tolerance at 150 mM salt in the hydroponics system. The 10 2490.1 plants tested showed segregation for salt tolerance with three significantly distinct groups present (FIG. 4). A high salt tolerant group and medium salt tolerant group and a salt intolerant group with a ratio of 1:2.5:1.5 respectively. The transgenic 2490.1 plants in Table 1 represent the high salt tolerant group whereas the null segregates represent the plants in the intolerant group.

Table 1 shows a greater than 3.5-fold increase in tiller number, head number and seed weight of the 2490.1 high salt segregates against the Westonia and 2490.1 low salt (intolerant) segregates. The high salt segregates were 4-fold higher for seed number than the two types of

TABLE 1
Tiller number, head number, seed number and seed weight for positive
transgenic plants for salt tolerance, commercial variety (Westonia)
and null segregates. S.D. is the standard deviation of the mean.
Transgenic 2490.1 has a Westonia background.

| Transgenic | | Null |
|---|---|---|
| 2490.1 | Westonia | segregates |

| Sample | Mean | S.D. | Mean | S.D. | Mean | S.D. |
|---|---|---|---|---|---|---|
| Tiller | 13.5 | 3.5 | 2.8 | 1.32 | 3.7 | 1.15 |
| number | | | | | | |
| Head | 11 | 1.4 | 2.6 | 1.17 | 3 | 0 |
| Number | | | | | | |
| Seed | 218 | 57.3 | 49.9 | 24.2 | 52.7 | 13.5 |
| number | | | | | | |
| Seed | 7.65 | 0.58 | 1.54 | 0.95 | 2.09 | 0.82 |
| weight | | | | | | |

controls.

### EXAMPLE 7

### Frost Tolerance Scoring

Seed from the T2 generation of plants containing the OAT transgene (lines 2490.1 and 2721.1) were planted 4 seeds per 4 L pot (containing sterile potting mix and fertiliser) and grown in a controlled environment room. The conditions within the room were 20° C. day (12 h, starting at 3:00 pm) and 14° C. night (12 h). Approximately 20 days prior to anthesis the temperature was ramped down in day temperature of 2° C. every second day and night temperature of 2° C. every third night to a final temperature of 8° C. day and 4° C. night.

Once anthesis had begun the plants were transferred to a frosting chamber (radiative chilling ramp and controlled floor/root temperature) where the temperature was dropped from 4° C. to −4° C. where the temperature was held for 3 hr before being ramped back to 4° C. This process occurred over a 12 hr period and once completed the plants were returned to the controlled environment room. The controlled environment room temperature was then increased for the day temperature at a rate of 2° C. every second day and night temperature of 4° C. every third day commencing seven days after the frost to a final temperature of 24° C. day and 18° C. night.

Thirteen days after frosting the transgenic plants and respective controls level of seed development was scored in relation to the wheat heads developmental stage. The scoring process used the Westonia non-transgenic plants that were not frosted as a base line (0 value) for the frosted Westonia plants and the transgenic line 2490.1 and the Carnamah not frosted plants as the base line for the frosted Carnamah and 2721.1. The scale for scoring was from 0 to 5 with 5 being little or no seed development in comparison to the control (base line).

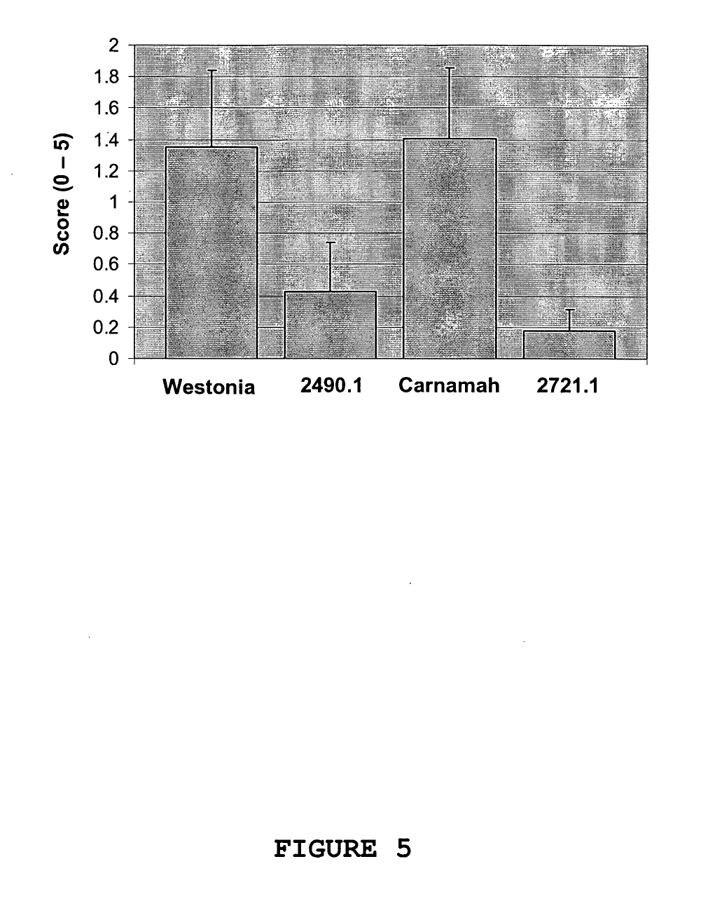

Both frosted transgenic lines showed a significant reduction in the level of impact on seed development (FIG. 5) in relation to the frosted controls (Westonia and Carnamah wheat varieties). On average the impact was over three fold less. Therefore these initial findings suggest that the transgenic plants are also frost tolerant.

### REFERENCE

Abdullah et al., 1986, Bio/Technology, 4: 1087-1090.
Benbrook et al., 1986, In: Proceedings Bio Expo 1986,
Butterworth, Stoneham, Mass., pp. 27-54.
Bevan et al., 1983, Nucleic Acids Res. 11:369-385.
Bower et al., 1996, Molecular Breeding, 2, 239-249.
Bytebier et al., 1987, Proc. Natl. Acad. Sci., USA, 84:5345-5349.
Callis et al., 1987, Genes and Development, 1:1183.
Capecchi, 1980, Cell, 22(2 Pt 2):479-88.
Cheng et al., 1998, Applied Biological Sciences, Vol. 95, Issue 6, 2767-2772.
Christensen & Quail, 1996, Transgenic Res., 5, 213-218.
Clapp, 1993, Clin. Perinatol. 20(1):155-168.
Corpet, 1988, Nucl. Acids Res., 16 (22), 10881-10890
Cristou et al., 1988, Plant Physiol, 87:671-674.
Curiel et al., 1991, Proc. Natl. Acad. Sci. USA, 88(19):8850-8854.
Curiel et al., 1992, Hum. Gen. Ther. 3(2):147-154.
Cushmore et al., 1983, Gen. Eng. of Plants, Plenum Press, New York, 29-38.
De la Pena et al., 1987, Nature, 235:274-276.
Eglitis & Anderson, 1988(a), Biotechniques, 6(7):608-614.
Eglitis et al., 1988(b), Adv. Exp. Med. Biol. 241:19-27.
Ellis et al. 1987, EMBO J. 6:11-16.
Fischhoff et al., 1987, Bio/Technology, 5:807-813.
Fraley et al., 1983, Proc. Natl. Acad. Sci., USA, 80:4803-4807.
Fraley et al., 1985, Biotechnology, 3:629.
Fromm et al., 1985, Proc. Natl. Acad. Sci. USA, 82:5824.
Fujimura et al., 1985, Plant Tissue Culture Letters, 2:74.
Fynan et al., 1993, Proc. Natl. Acad. Sci. USA, 90(24): 11478-11482.
Gallie et al., 1989, Plant Cell, 1:301-311.
Graham & van der Eb, 1973, Virology, 54(2):536-539.
Herrera-Estrella et al., 1983, Nature, 303:209.
Hess, 1987, Intern Rev. Cytol., 107:367.
Horsch et al., 1985, Science, 227:1229-1231.
Ishida et al., 1996, Nature Biotechnology, 14 (6):745-50.
Johnston & Tang, 1994, Methods Cell. Biol. 43(A):353-365.
Jorgensen et al., 1987, Mol. Gen. Genet., 207:471-477.
Joshi, 1987, Nucl. Acids Res., 15:6643-6653.
Kawata et al., 1988
Kay et al., 1987, Science, 236:1299-1302.
Keller et al., 1989, The EMBO Journal, 8:1309-1314.
Klee et al., 1985, In: Plant DNA Infectious Agents, Hohn & Schell, eds., Springer-Verlag, New York pp. 179-203.
Klein et al., 1987, Nature, 327:70.
Klein et al., 1988, Proc. Natl. Acad. Sci., USA, 85:8502-8505.
Langridge et al., 1989, Proc. Natl. Acad. Sci. U.S.A., 86:3219-3223.
Lorz et al., 1985, Mol. Gen. Genet., 199:178.
Lu et al., 1993, J. Exp. Med. 178(6):2089-2096.
Ma et al., 1988, Nature, 334:631-633.
Macejak & Sarnow, 1991, Nature, 353:90-94.
Marcotte et al., 1988, Nature, 335:454.
McBride and Summerfelt, 1989
McElroy et al., 1990, Plant Cell. 2: 163171.
Medberry et al., 1992, The Plant Cell, 4: 185-192.
Murashige & Skoog, 1962, Physiol. Plant. 15:473-497.
Neuhaus et al., 1987, Theor. Appl. Genet., 75:30.
Omirulleh et al., 1993, Plant Molecular Biology, 21:415-428.
Pelletier & Sonenberg, 1985, Mol. Cell. Biol. 5:3222-3230.
Pelletier & Sonenberg, 1988, Nature, 334: 320-325.
Potrykus et al., 1985, Plant Molecular Biology Reporter, 3:117-128.
Poulsen et al., 1986, Mol. Gen. Genet., 205:193-200.
Rogers & Bendich, 1988, Plant Mol. Biol. A6: 1-10.
Simpson et al., 1986, Science, 233:34-38.
Spielmann et al., 1988, Nucleic Acids Res., 16:1199.
Toriyama et al., 1987, Plant Sci., 48: 123-128.
Uchimiya et al., 1986, Mol. Gen. Genet. 204: 204-207.
Van Tunen et al., 1988, EMBO J., 7:1257.
Vasil et al., 1989, Plant Physiol 91: 15751579.
Vogel et al., 1989, J. Cell Biochem., (supplement 13D, 312).
Wagner et al., 1992, Proc. Natl. Acad. Sci. USA, 89(13):6099-6103.
Weeks et al., 2000, Crop Science, 40: 1749-1754.
Weissbach & Weissbach, 1988, In: Methods for Plant Molecular Biology, Academic Press, Inc., San Diego, Calif.
Wenzler et al., 1989, Plant Mol. Biol., 12:41-50.
Winter et al., 1988, Mol. Gen. Genet., 221(2):315-319.
Wong & Neumann, 1982, Biochim. Biophys. Res. Commun. 107(2):584-587.
Yamada et al., 1986, Plant Cell Rep., 4:85.
Yang et al., 1990, Proc. Natl. Acad. Sci., USA, 87:4144-4148.
Zhou et al., 1983, Methods in Enzymology, 101:433.

1. A stress tolerant wheat plant, wherein said wheat plant has been transformed with a nucleic acid molecule, which codes for ornithine amino transferase (OAT).

2. A stress tolerant wheat plant according to claim 1, wherein the nucleic acid molecule is a cDNA molecule having a nucleotide sequence which is substantially that shown in FIG. 2 (SEQ ID NO:1) or biologically active fragment thereof.

3. A stress tolerant wheat plant according to claim 1, wherein the plant has an increased capacity to withstand a stress selected from the group consisting of drought stress, salt stress, dehydration stress, heat stress, cold stress, freezing stress, water logging stress, wounding stress, mechanical stress, oxidative stress, ozone stress, high light stress, heavy metals stress, nutrient deprivation stress and toxic chemical stress when compared to untransformed wheat plants.

4. A method for protecting a wheat plant from stress, comprising the step of introducing a nucleic acid molecule into a wheat plant, which nucleic acid molecule codes for ornithine amino transferase (OAT).

5. A method according to claim 4, wherein the stress is selected from the group consisting of drought, salt, dehydration, heat, cold, freezing, water logging, wounding, mechanical stress, oxidative stress, ozone, high light, heavy metals, nutrient deprivation and toxic chemicals.

6. A method according to claim 4, wherein the stress is salt or drought.

7. A method according to claim 4, wherein the stress is the presence of more than 100 mM salt.

8. A method according to claim 4, wherein the ornithine amino transferase (OAT) nucleic acid molecule is isolated from Arabidopsis thaliana.

9. A method according to claim 4, wherein the wheat plant is selected from the group consisting of Triticum aestivum, and Triticum durum.

10. A transgenic wheat plant, plant material, seeds or progeny thereof, comprising a nucleic acid molecule, which codes for ornithine amino transferase (OAT), wherein the expression of said nucleic acid molecule results in a transgenic plant, plant material, seeds or progeny thereof which is capable of growing in the presence of more than 100 mM salt.

11. A nucleic acid construct comprising a promoter isolated from a plant and an ornithine amino transferase (OAT) gene, wherein said construct is capable of transforming a wheat plant such that said wheat plant becomes stress tolerant.

12. A nucleic acid construct according to claim 11, wherein said promoter is a constitutive promoter, a ubiquitous promoter, a stress-inducible promoter, a tissue-specific promoter or a developmentally controlled promoter.

13. A nucleic acid construct according to claim 11, wherein said promoter is the ubiquitin promoter.

14. A nucleic acid construct according to claim 11, wherein the nucleic acid molecule is a cDNA molecule having a nucleotide sequence which is substantially that shown in FIG. 2 (SEQ ID NO:1) or biologically active fragment thereof.

15. A method for producing a transgenic wheat plant with an induced or increased tolerance to salt, the method comprising the steps of:
a) transforming plant tissue or cell of a wheat plant with a nucleic acid molecule which codes for ornithine amino transferase (OAT);
b) regenerating the tissue or cell into a whole plant, and
c) expressing the OAT in the regenerated plant for a time and under conditions sufficient to induce or increase the tolerance of the plant to greater than 100 mM salt.


SEQUENCE LISTING (2 sequences)

SEQ ID NO: 1 - DNA, 1457 bp, Arabidopsis thaliana
gatccgcttcacaatggcagccaccacgagacggttgctttactatgtgtccaaaagattctctaccgccggagtgcggcggagctatggcggtctgccacaatcgaattccaaatctcctccgtcttcttctcaacgattgatggaattggaatctgaattcagtgcccacaattaccatcctgttccggttgtgttttctcgcgcgaatggctcaactatatgggaccctgaaggaaagagatacattgattttcttgcagcttactctgcagttaatcagggacattgtcatcctaagatcatgaaggcattgcaggaacaagtggagaagctcactttaagttcacgagcgttttacaacgataaattcccggtatttgctgagcgtctcacaaacatgttcggttatgatatggtgcttcctatgaacacgggtgctgaaggtgtcgaaactgctttgaaactagcaagaaaatggggtcatgaaaagaaaaatattcccaaagacgaggctatcattgtctcttgttgtggctgctttcatggacgtacattagcaattgtttccatgagttgtgacaatgatgctactcgtggattcgggccattgttgccagggaatcttaaagttgattttggtgatgcggattcacttgagaaaatctttaaagaaaagggagatagaatagcgggatttctattcgagcctattcaaggcgaagctggagttattattcctcccgacggttacttgaaagctgttagagaactctgcacaaaatacaatgttttgatgatagcggatgaagtacaaagcggtctggctagatccgggaagatgctagcttgtgattgggaagaaattcgtcctgacatggtgatacttgggaaagcattaggtggaggagtaattccagtaagtgcagtgcttgctgataaagacgtgatgcttcatataaaacctggtcaacacggaagcacatttggtggtaaccctttagctagtgctgtagctatggcttctttagacgttatcgtggaagaaaaactcgttgaaagatcagcaagtcttggagaggaactgaggattcaattgaatgaaatcaagaaacagtttccgaagtacataaaggaagtacgaggaagaggcttgtttaatgcgattgagttcaatagcgaaagcttgtccccggtttcagcttacgatatttgcttgagcttgaaggagagaggagtcttggctaaacctactcacaacactattgtccggttaactccaccactctctataagctctgatgaactccgagatggatctgaggctcttcacgatgtactcgagctcgatcttccaaatctgctgaaaatcaattccggtaaaactccggtttctcatataaccgagtgtgatcgctgtggaagaaacctctatgcttgaacccagctttcggtac

SEQ ID NO: 2 - protein, 475 aa, Arabidopsis thaliana
MAATTRRLLYYVSKRFSTAGVRRSYGGLPQSNSKSPPSSSQRLMELESEFSAHNYHPVPVVFSRANGSTIWDPEGKRYIDFLAAYSAVNQGHCHPKIMKALQEQVEKLTLSSRAFYNDKFPVFAERLTNMFGYDMVLPMNTGAEGVETALKLARKWGHEKKNIPKDEAIIVSCCGCFHGRTLAIVSMSCDNDATRGFGPLLPGNLKVDFGDADSLEKIFKEKGDRIAGFLFEPIQGEAGVIIPPDGYLKAVRELCTKYNVLMIADEVQSGLARSGKMLACDWEEIRPDMVILGKALGGGVIPVSAVLADKDVMLHIKPGQHGSTFGGNPLASAVAMASLDVIVEEKLVERSASLGEELRIQLNEIKKQFPKYIKEVRGRGLFNAIEFNSESLSPVSAYDICLSLKERGVLAKPTHNTIVRLTPPLSISSDELRDGSEALHDVLELDLPNLLKINSGKTPVSHITECDRCGRNLYA